(19) United States
(12) Patent Application Publication
(10) Pub. No.: US 2019/0167174 A1
(43) Pub. Date: Jun. 6, 2019

(54) Computer Program for Balancing Mind

(71) Applicant: Miross Institute, Inc., Kobe-shi, Hyogo (JP)

(72) Inventor: Hiroshi Matsumoto, Hyogo (JP)

(73) Assignee: Miross Institute, Inc., Kobe-shi, Hyogo (JP)

(21) Appl. No.: 16/324,225

(22) PCT Filed: Aug. 31, 2017
(86) PCT No.: PCT/JP2017/031384

§ 371 Date: Feb. 8, 2019

(30) Foreign Application Priority Data
Aug. 31, 2016 (JP) 2016-169334

(51) Int. Cl. A61B 5/16; A61B 5/00; G09B 5/06; G09B 5/02

(52) U.S. Cl. CPC A61B 5/165; A61B 5/748; A61B 5/7435; G09B 5/02; A61B 5/7405; G09B 5/067

(57) ABSTRACT

A computer program for achieving balance of the mind of an examinee is provided. A computer program configured to cause a computer including a CPU and a memory to execute processing for achieving balance of the mind of an examinee, the computer program being configured to cause the computer to execute the following steps:
a first step of making the examinee visualize a set of symbols;
a second step of applying the symbols to a feeling of the examinee and a state of the examinee's mind; and
a third step of making the examinee understand a recognition system represented by the set.

### TECHNICAL FIELD

The present invention relates to a computer program for achieving balance of the mind of an examinee (a subject), and particularly relates to a computer program for achieving balance of the mind of an examinee by using a set of symbols.

### BACKGROUND ART

The number of people with mental disorders is recently increasing. In social life, it is becoming more difficult to maintain balance of the mind. Patent Literature 1 discloses a measure for resolving mental problems by a method of inducing various states of consciousness by entraining brain waves into unique waveforms characteristic of specific states of consciousness by modulating the binaural beat signals with brain waves associated with particular activities.

### CITATION LIST

### Patent Literature

PTL 1: U.S. Pat. No. 5,213,562

### SUMMARY OF INVENTION

### Technical Problem

The measure disclosed in Patent Literature 1 induces various states of consciousness by working on the examinee's auditory sense. In contrast, an object of the present invention is to provide a computer program for achieving balance of the examinee's mind by using a set of symbols.

### Solution to Problem

To achieve the above object, the present invention provides the computer programs described in the following items.

### Item 1.

A computer program configured to cause a computer including a processing device and a storage device to execute processing for achieving balance of the mind of an examinee, the computer program being configured to cause the computer to execute the following steps:
a first step of making the examinee visualize a set of symbols;
a second step of applying the symbols to a feeling of the examinee and a state of the examinee's mind; and
a third step of making the examinee understand a recognition system represented by the set.

### Item 2.

The computer program according to Item 1, wherein the computer is a robot having artificial intelligence.

### Item 3.

The computer program according to Item 1 or 2,
wherein the set includes:
a first symbol;
a second symbol located obliquely below the first symbol;
a third symbol that has a shape representing a concept in contrast with that of the second symbol, and that is a mirror image of the second symbol in terms of the position with respect to an imaginary vertical line passing through the first symbol;
a fourth symbol that has a shape in common with that of the third symbol and that is located obliquely below the second symbol; and
a fifth symbol that has a shape in common with that of the second symbol, and that is a mirror image of the fourth symbol in terms of the position with respect to an imaginary vertical line passing through the second symbol;
wherein in the second step, the second symbol is associated with the examinee's thought, the third symbol is associated with an object of the examinee's thought, the fourth symbol is associated with the examinee's unconsciousness, and the fifth symbol is associated with a blind spot in the examinee's mind; and
wherein in the third step, the examinee is made to understand the recognition system by an explanation based on contrasting concepts between the second symbol and the third symbol, and between the fourth symbol and the fifth symbol, the explanation being that the blind spot in the examinee's mind associated with the fifth symbol represents a concept identified with by the examinee's thought associated with the second symbol and produces the examinee's unconsciousness associated with the fourth symbol, and that the feeling from the object associated with the third symbol is a projection of the examinee's unconsciousness associated with the fourth symbol.

### Item 4.

The computer program according to Item 3, wherein the second, fifth, and seventh symbols have a “+” shape while the third, fourth, and sixth symbols have a “−” shape.

### Item 5.

The computer program according to Item 3 or 4,
wherein the set further includes:
a sixth symbol that has a shape in common with that of the third symbol and that is located obliquely below the third symbol; and
a seventh symbol that has a shape in common with that of the second symbol, and that is a mirror image of the sixth symbol in terms of the position with respect to an imaginary vertical line passing through the third symbol;

wherein in the second step, the sixth symbol is associated with a blind spot in the object's mind, and the seventh symbol is associated with the object's unconsciousness; and
wherein in the third step, the examinee is given an explanation that the blind spot in the object's mind associated with the sixth symbol represents a concept identified with by the object's thought associated with the third symbol and produces the object's unconsciousness associated with the seventh symbol, and that the feeling from the examinee associated with the second symbol is a projection of the object's unconsciousness associated with the seventh symbol.

### Advantageous Effects of Invention

The present invention provides a computer program for achieving balance of the mind of an examinee. The present invention can be implemented using a computer without human intervention of a counselor or the like and thus allows an examinee to achieve balance of the mind in a simple manner as if the examinee were playing a game.

### BRIEF DESCRIPTION OF DRAWINGS

FIG. 1 is a block diagram of a computer used in one embodiment of the present invention.

FIG. 2 is a set of symbols used in one embodiment of the present invention.

FIG. 3 is a modification of the above set.

FIG. 4 is a modification of the above set.

FIG. 5 is a modification of the above set.

FIG. 6 is a flow chart illustrating the entire processing of a method for achieving balance of the mind of an examinee, performed by the computer shown in FIG. 1 executing a computer program.

FIG. 7 is a flow chart illustrating a specific procedure of step S1 shown in FIG. 6.

FIG. 8 is a flow chart illustrating a specific procedure of step S2 shown in FIG. 6.

FIG. 9 shows an example of screen display in step S2.

FIG. 10 shows an example of screen display in step S2.

FIG. 11 is a flow chart illustrating a part of a specific procedure of step S3 shown in FIG. 6.

FIG. 12 shows an example of screen display in step S3.

FIG. 13 shows an example of screen display in step S3.

FIG. 14 shows an example of screen display in step S3.

FIG. 15 shows an example of screen display in step S3.

FIG. 16 shows an example of screen display in step S3.

FIG. 17 shows an example of screen display in step S3.

FIG. 18 shows an example of screen display in step S3.

FIG. 19 shows an example of screen display in step S3.

FIG. 20 shows an example of screen display in step S3.

FIG. 21 shows an example of screen display in step S3.

FIG. 22 is a flow chart illustrating a part of the specific procedure of step S3 shown in FIG. 6.

FIG. 23 shows an example of screen display in step S3.

FIG. 24 shows an example of screen display in step S3.

FIG. 25 shows an example of screen display in step S3.

FIG. 26 shows an example of screen display in step S3.

FIG. 27 shows an example of screen display in step S3.

FIG. 28 is a flow chart illustrating a part of the specific procedure of step S3 shown in FIG. 6.

FIG. 29 shows an example of screen display in step S3.

FIG. 30 shows an example of screen display in step S3.

FIG. 31 shows an example of screen display in step S3.

FIG. 32 shows an example of screen display in step S3.

FIG. 33 shows an example of screen display in step S3.

FIG. 34 shows an example of screen display in step S3.

FIG. 35 shows an example of screen display in step S3.

FIG. 36 shows an example of screen display in step S3.

FIG. 37 shows an example of screen display in step S3.

FIG. 38 shows an example of screen display in step S3.

FIG. 39 shows an example of screen display in step S3.

FIG. 40 shows an example of screen display in step S3.

FIG. 41 is a flow chart illustrating a part of the specific procedure of step S3 shown in FIG. 6.

FIG. 42 shows an example of screen display in step S3.

FIG. 43 shows an example of screen display in step S3.

FIG. 44 shows an example of screen display in step S3.

FIG. 45 is a flow chart illustrating a part of the specific procedure of step S3 shown in FIG. 6.

FIG. 46 shows an example of screen display in step S3.

FIG. 47 shows an example of screen display in step S3.

FIG. 48 shows an example of screen display in step S3.

FIG. 49 shows an example of screen display in step S3.

FIG. 50 shows an example of screen display in step S3.

FIG. 51 shows an example of screen display in step S3.

FIG. 52 shows an example of screen display in step S3.

FIG. 53 shows an example of screen display in step S3.

FIG. 54 shows an example of screen display in step S3.

FIG. 55 shows an example of screen display in step S3.

FIG. 56 shows an example of screen display in step S3.

FIG. 57 shows an example of screen display in step S3.

FIG. 58 is a flow chart illustrating a part of the specific procedure of step S3 shown in FIG. 6.

FIG. 59 shows an example of screen display in step S3.

FIG. 60 shows an example of screen display in step S3.

FIG. 61 shows an example of screen display in step S3.

FIG. 62 shows an example of screen display in step S3.

FIG. 63 shows an example of screen display in step S3.

FIG. 64 shows an example of screen display in step S3.

FIG. 65 shows an example of screen display in step S3.

FIG. 66 shows an example of screen display in step S3.

FIG. 67 shows an example of screen display in step S3.

FIG. 68 shows an example of screen display in step S3.

FIG. 69 is a flow chart illustrating a part of the specific procedure of step S3 shown in FIG. 6.

FIG. 70 shows an example of screen display in step S3.

FIG. 71 shows an example of screen display in step S3.

FIG. 72 shows an example of screen display in step S3.

FIG. 73 shows an example of screen display in step S3.

FIG. 74 shows an example of screen display in step S3.

FIG. 75 shows an example of screen display in step S3.

FIG. 76 shows an example of screen display in step S3.

FIG. 77 shows an example of screen display in step S3.

FIG. 78 shows modifications of the first symbol.

FIG. 79 shows graphs that indicate results of the Example of the present invention.

### DESCRIPTION OF EMBODIMENTS

The following describes embodiments of the present invention with reference to accompanying drawings. However, the present invention is not limited to the following embodiments.

### 1. Computer

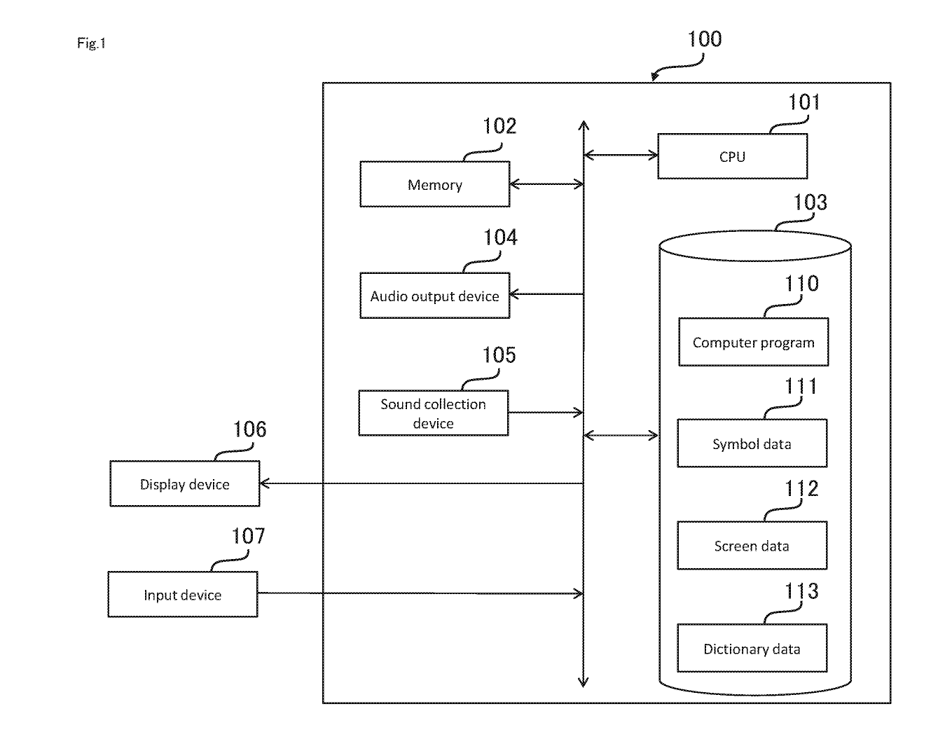

FIG. 1 is a block diagram of a computer 100 used in one embodiment of the present invention. In this embodiment, it is preferable that the computer 100 includes a robotic computer with artificial intelligence that has an appearance similar to humans; however, the computer 100 may include, for example, a general-purpose personal computer, a tablet device, a smartphone, a VR device, or the like. The computer 100 may be provided on a cloud.

As shown in FIG. 1, the computer 100 includes a CPU (processing device) 101, a memory (storage device) 102, an auxiliary storage device 103, an audio output device 104, a sound collection device 105, etc., and these are connected to each other via a bus. The computer 100 is connected to a display device 106 and an input device 107 in a wired or wireless manner.

The CPU 101 reads a computer program 110 stored in the auxiliary storage device 103 into the memory 102 and executes it. The computer 100 thereby executes steps as described below for achieving balance of the mind of an examinee.

The memory 102 is a storage device into which data can be written and read at high speed, such as DRAM or SRAM. The auxiliary storage device 103 is, for example, a mass-storage device, such as an SSD or HDD. The auxiliary storage device 103 stores programs, symbol data 111 described later, which includes a set 10 of symbols, screen data 112 for displaying question screens etc., and dictionary data 113, in which tams are recorded. The audio output device 104 includes a speaker, and the sound collection device 105 includes a microphone.

The computer program 110 according to the present invention is stored as a part of data of the auxiliary storage device 103 by installing the computer program 110 onto the computer 100. The computer 100 may be connected to a communication network, such as the Internet, and the computer program 110 may be downloaded via the communication network. The computer program 110 may be installed onto the computer 100 via a computer-readable storage medium, such as CD-ROM.

The display device 106 includes, for example, a liquid crystal display, and displays, for example, a set of symbols described later. The input device 107 includes, for example, a touch panel or a keyboard. An examinee can instruct the computer 100 and respond to the computer 100 by operating the input device 107.

### 2. Set of Symbols

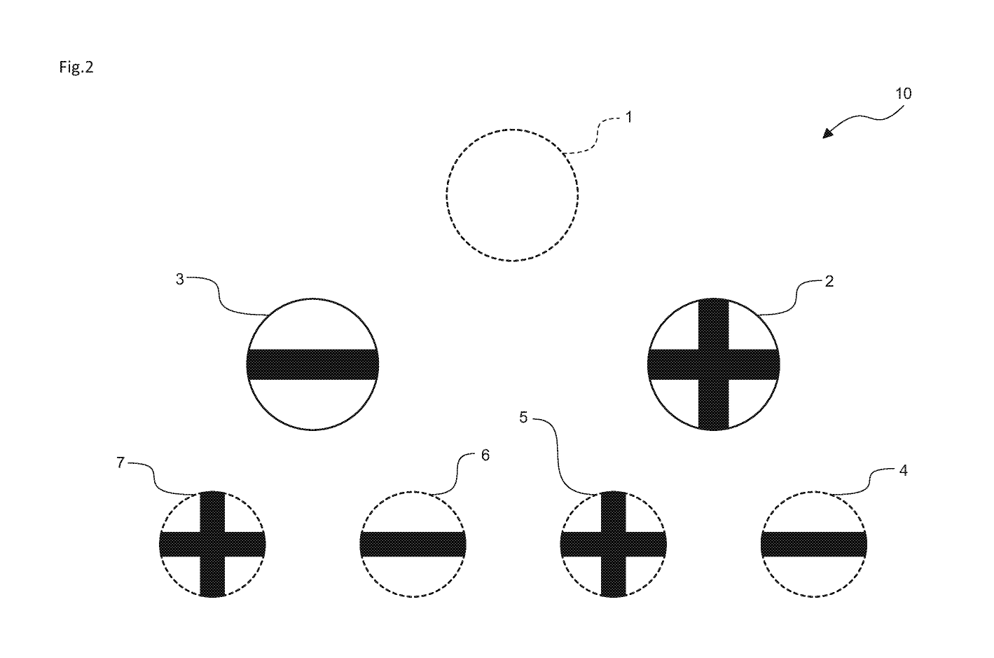

The present invention has a feature of using a set of symbols to achieve balance of the examinee's mind. FIG. 2 shows the set 10 of symbols (hereinafter referred to as a “set 10”) used in one embodiment of the present invention.

The set 10 includes first to seventh symbols 1 to 7. The first to seventh symbols 1 to 7 are, as a whole, located in mirror-image positions. As described later, the set 10 is shown to an examinee on the display device 106 shown in FIG. 1, but the way the set 10 is shown to an examinee is not limited to this. The set 10 may be displayed on a display medium, such as a whiteboard, Google Glass (registered trademark), a paper medium, a monitoring screen, or a VR screen.

The first symbol 1 (which hereinafter may be simply referred to as “symbol 1”) is located in the center of the uppermost part of the set 10. The shape and pattern of the first symbol 1 are not particularly limited. However, the first symbol 1 does not have, at least, a shape or pattern that refers to an image biased to either of two contrasting concepts, such as “+” and “−.”

The second symbol 2 (which hereinafter may be simply referred to as “symbol 2”) is located obliquely below the first symbol 1. In this embodiment, the second symbol 2 is located obliquely to the right and downward from the first symbol 1 with a space therebetween, and has a “+” shape.

The third symbol 3 (which hereinafter may be simply referred to as “symbol 3”) has a shape representing a concept in contrast with that of the second symbol 2, and is a mirror image of the second symbol 2 in terms of the position with respect to an imaginary vertical line passing through the first symbol 1. In this embodiment, the third symbol 3 has a “−” shape and is located obliquely to the left and downward from the first symbol 1 with a space therebetween.

The fourth symbol 4 (which hereinafter may be simply referred to as “symbol 4”) has a shape in common with that of the third symbol 3, and is located obliquely below the second symbol 2. In this embodiment, the fourth symbol 4 has a “−” shape and is located obliquely to the right and downward from the second symbol 2 with a space therebetween.

The fifth symbol 5 (which hereinafter may be simply referred to as “symbol 5”) has a shape in common with the second symbol 2, and is a mirror image of the fourth symbol 4 in terms of the position with respect to an imaginary vertical line passing through the second symbol 2. In this embodiment, the fifth symbol 5 has a “+” shape and is located obliquely to the left and downward from the second symbol 2 with a space therebetween.

The sixth symbol 6 (which hereinafter may be simply referred to as “symbol 6”) has a shape in common with that of the third symbol 3, and is located obliquely below the third symbol 3. In this embodiment, the sixth symbol 6 has a “−” shape and is located obliquely to the right and downward from the third symbol 3 with a space therebetween.

The seventh symbol 7 (which hereinafter may be simply referred to as “symbol 7”) has a shape in common with that of the second symbol 2, and is a mirror image of the sixth symbol 6 in terms of the position with respect to an imaginary vertical line passing through the third symbol 3.

In this embodiment, the seventh symbol 7 has a “+” shape and is located obliquely to the left and downward from the third symbol 3 with a space therebetween.

As above, the shape of the second, fifth, and seventh symbols 2, 5, and 7 represents a concept in contrast with that of the shape of the third, fourth, and sixth symbols 3, 4, and 6. In this embodiment, the second, fifth, and seventh symbols 2, 5, and 7 have a “+” shape while the third, fourth, and sixth symbols 3, 4, and 6 have a “−” shape.

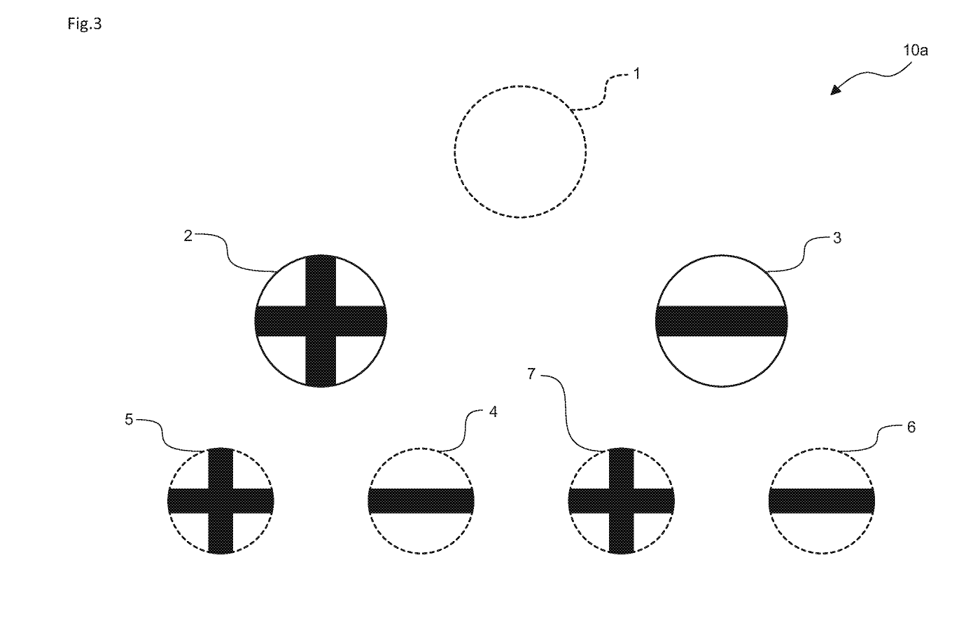

The set used in the present invention is not limited to the set 10 shown in FIG. 2. FIGS. 3 to 5 show modifications of the set 10.

For example, the second symbol 2 and the third symbol 3 may be replaced with each other, as shown in the set 10a of FIG. 3. Further, in the set 10a, with the replacement between the second symbol 2 and the third symbol 3, the fourth and fifth symbols 4 and 5 and the sixth and seventh symbols 6 and 7 are replaced with each other.

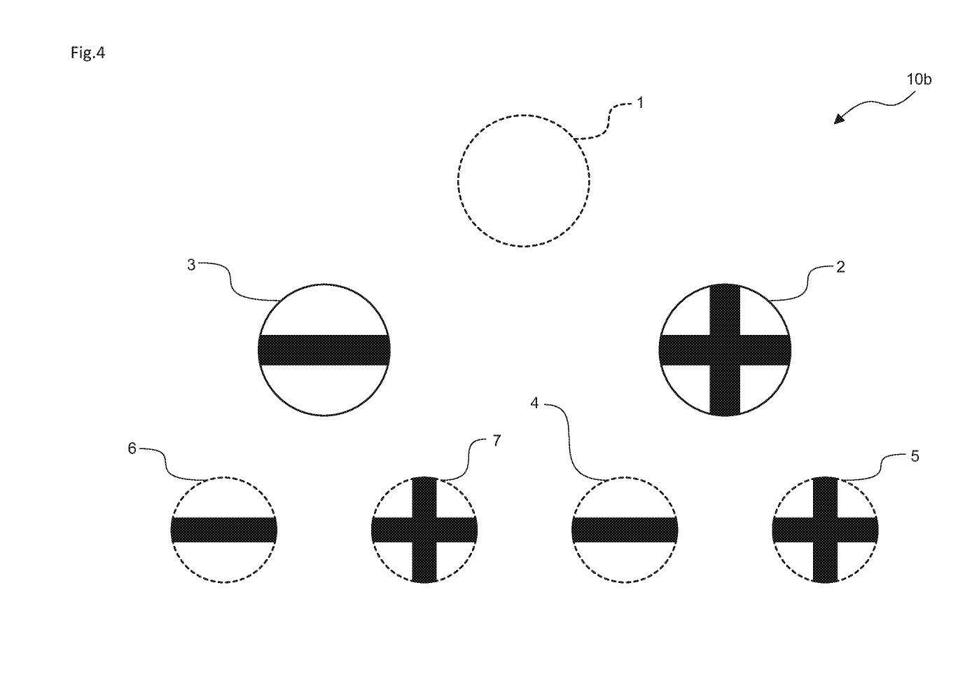

In the set 10b shown in FIG. 4, the fourth symbol 4 and the fifth symbol 5 of the set 10 shown in FIG. 2 are replaced with each other while the sixth symbol 6 and the seventh symbol 7 are replaced with each other.

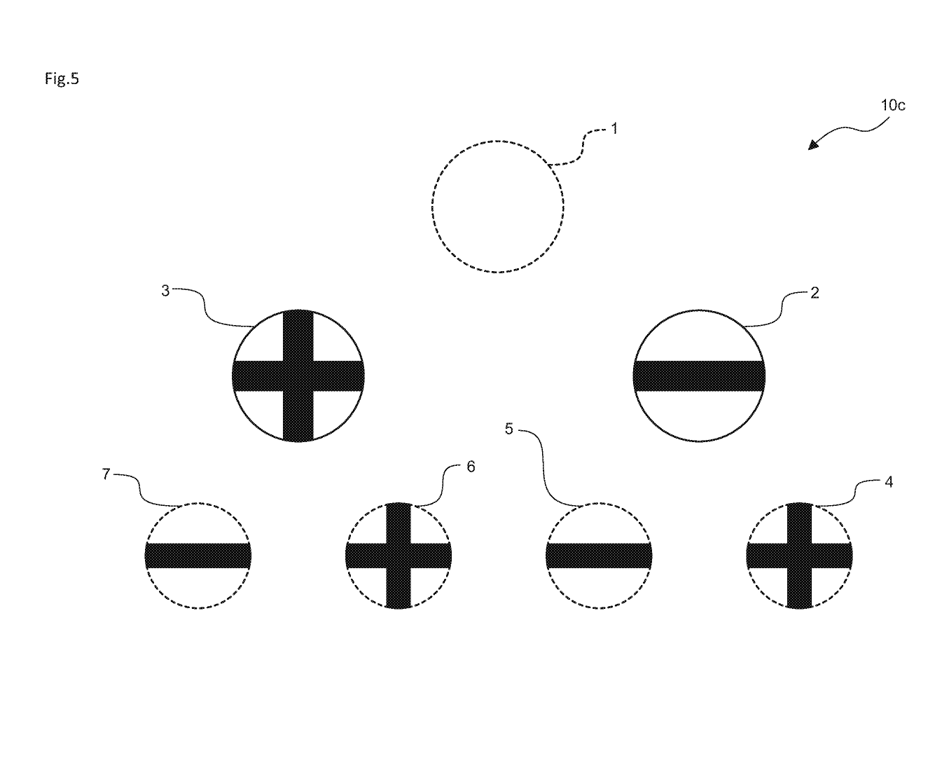

In the set 10c shown in FIG. 5, the second to seventh symbols 2 to 7 of the set 10 shown in FIG. 2 are each made to have a shape representing the opposite concept.

Specifically, in the set 10c, the second, fifth, and seventh symbols 2, 5, and 7 have a “−” shape while the third, fourth, and sixth symbols 3, 4, and 6 have a “+” shape.

In the second and third symbols 2 and 3, the “+” or “−” sign is surrounded by a solid-line circle. In the fourth to seventh symbols 4 to 7, the “+” or “−” sign is surrounded by a dotted-line circle. The solid line surrounding the second and third symbols 2 and 3 indicates that the second and third symbols 2 and 3 respectively correspond to the examinee's thought and an object of the examinee's thought. The dotted line surrounding the fourth to seventh symbols 4 to 7 indicates that these symbols correspond to a state of the examinee's mind that is not exposed. “State of mind” specifically refers to the unconsciousness and blind spots in the mind of an examinee, as well as the unconsciousness and blind spots in the mind of an object of the examinee's thought.

As described above, in the sets 10, 10a, 10b, and 10c according to the present invention, the first to seventh symbols 1 to 7, as a whole, are located bisymmetrically. Further, the second, fifth, and seventh symbols 2, 5, and 7 have a shape representing a concept in contrast with that of the third, fourth, and sixth symbols 3, 4, and 6.

In the examples shown in FIGS. 2 to 5, the second to seventh symbols 2 to 7 have a “+” or “−” shape. However, in the present invention, the shapes are not limited to these shapes as long as they represent concepts in contrast with each other. For example, “♂” and “♀,” “O” and “X,” “↑” and “↓,” and “←,” and “→,” may be used. A pair of corresponding concepts may be applied to these contrasting shapes to implement the recognition system.

The shape and pattern of the first symbol 1 are different from those of the second to seventh symbols 2 to 7. The shape and pattern of the first symbol 1 are not particularly limited as long as they do not refer to an image biased to either of the two contrasting concepts. In this embodiment, the first symbol 1 is a dotted-line circular frame.

### 3. Procedure of Method

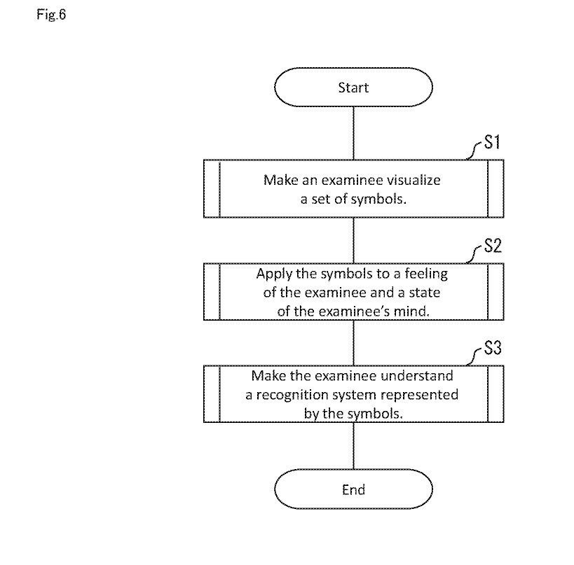

FIG. 6 is a flow chart illustrating the entire processing for achieving balance of the mind of an examinee, performed by the computer 100 shown in FIG. 1 executing the computer program 110. The processing comprises step S1 of making the examinee visualize a set of symbols, step S2 of applying the symbols to a feeling of the examinee and a state of the examinee's mind, and step S3 of making the examinee understand a recognition system represented by the set. The following describes the above method when performed using the set 10 shown in FIG. 2.

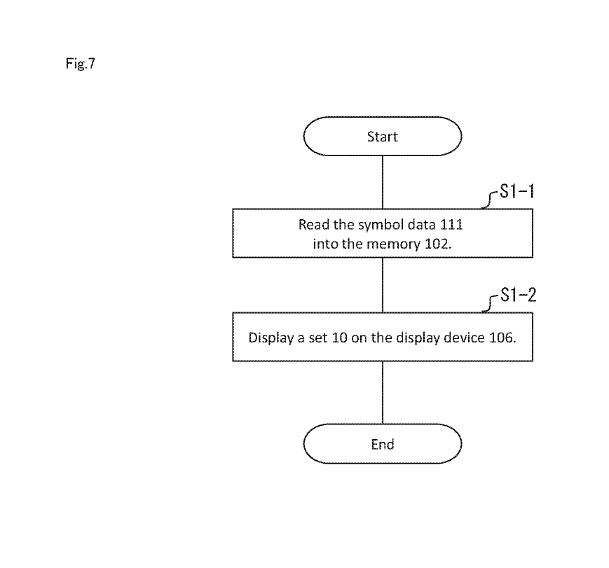

In step S1, the computer 100 makes an examinee visualize the set 10. In this embodiment, as shown in FIG. 7, the CPU 101 reads the symbol data 111 into the memory 102 according to the algorithm of the computer program 110 (step S1-1), and the set 10 is displayed on the display device 106 (step S1-2). The examinee is thereby made to visualize the set 10. The method of showing the set 10 is not particularly limited. For example, the robotic computer 100 having artificial intelligence may show the examinee a paper medium on which the set 10 is depicted, or a display medium, such as a whiteboard, Google Glass (registered trademark), a projector, a monitoring screen, a tablet screen, or a VR screen, may be used to display the set 10 and show it to the examinee. Alternatively, the size, shape, pattern, arrangement, etc. of each symbol in the set 10 may be explained to the examinee by voice to make the examinee visualize the set 10.

Subsequently, in step S2, the computer 100 applies the symbols of the set 10 to an examinee's feeling and a state of the examinee's mind. Specifically, the second symbol 2 is associated with the examinee's thought, the third symbol 3 is associated with an object of the examinee's thought, the fourth symbol 4 is associated with the examinee's unconsciousness, the fifth symbol 5 is associated with a blind spot in the examinee's mind, the sixth symbol 6 is associated with a blind spot in the object's mind, and the seventh symbol 7 is associated with the object's unconsciousness. “Examinee's thought” as used herein does not refer to the entirety of thought of the examinee but to a thought that produces conflict in the mind of the examinee.

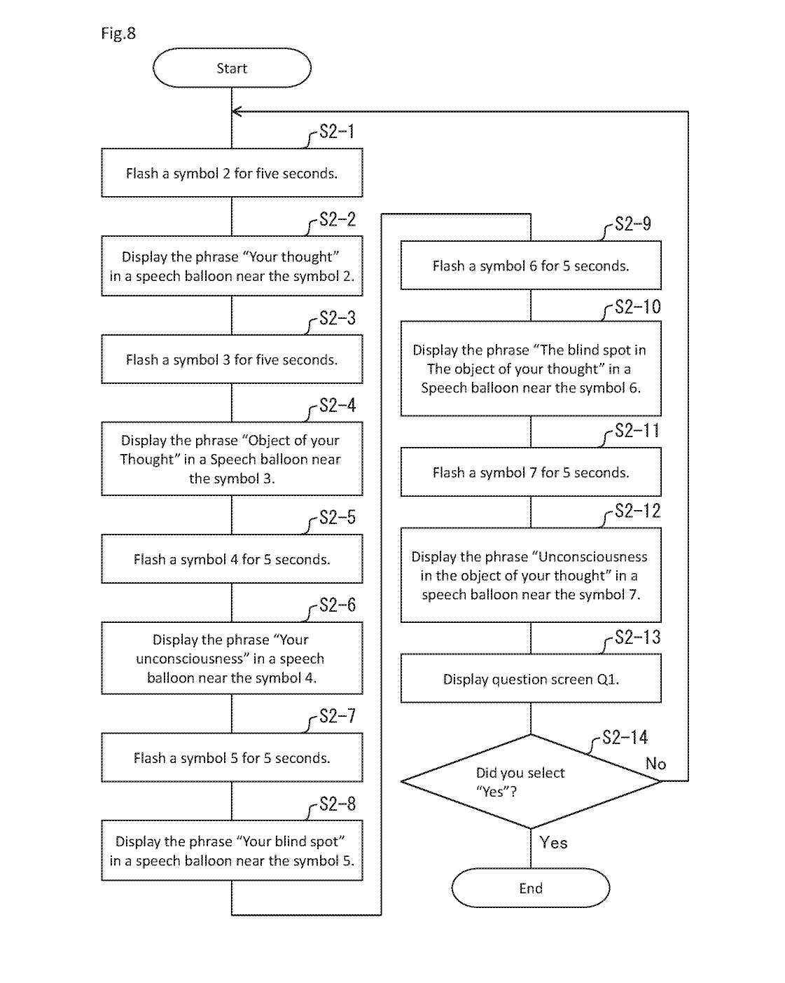

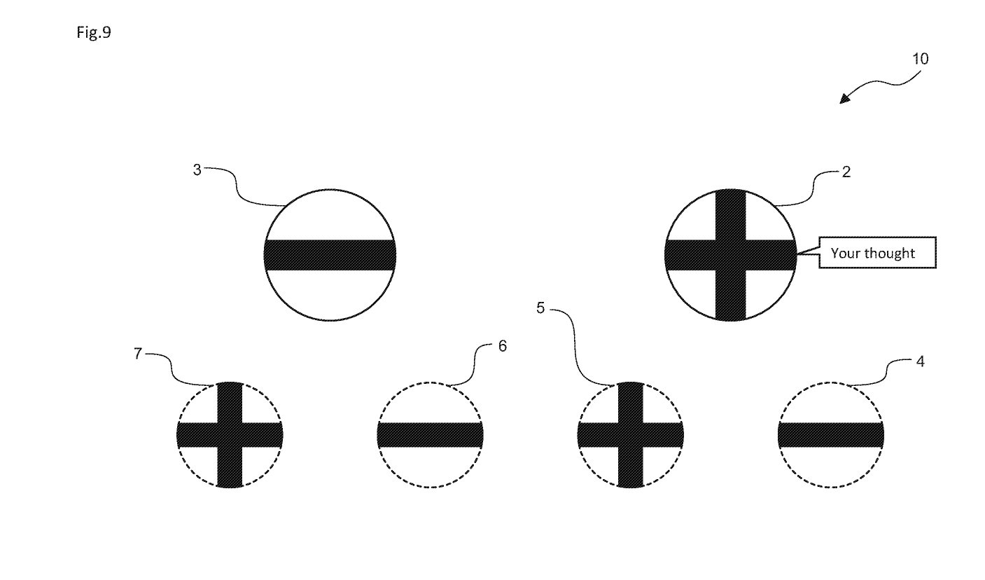

A specific example of the procedure in step S2 of this embodiment is described below. FIG. 8 is a flow chart illustrating a specific procedure of step S2 shown in FIG. 6. First, the second symbol 2 is flashed for 5 seconds in a state where the set 10 is displayed on the display device 106 (step S2-1). Thereafter, as shown in FIG. 9, the phrase “Your thought” is displayed in a speech balloon near the second symbol 2.

A similar procedure is performed for the third to seventh symbols 3 to 7. Specifically, in step S2-3 of FIG. 8, the third symbol 3 is flashed for 5 seconds, and then the phrase “Object of your thought” is displayed in a speech balloon near the third symbol 3 (step S2-4). Furthermore, the fourth symbol 4 is flashed for 5 seconds (step S2-5), and then the phrase “Your unconsciousness” is displayed in a speech balloon near the fourth symbol 4 (step S2-6). Furthermore, the fifth symbol 5 is flashed for 5 seconds (step S2-7), and then the phrase “Your blind spot” is displayed in a speech balloon near the fifth symbol 5 (step S2-8). Furthermore, the sixth symbol 6 is flashed for 5 seconds (step S2-9), and then the phrase “The blind spot in the object of your thought” is displayed in a speech balloon near the sixth symbol 6 (step S2-10). Furthermore, the seventh symbol 7 is flashed for 5 seconds (step S2-11), and then the phrase “Unconsciousness in the object of your thought” is displayed in a speech balloon near the seventh symbol 7 (step S2-12). The symbols are applied to a feeling of the examinee and a state of the examinee's mind by displaying such a screen.

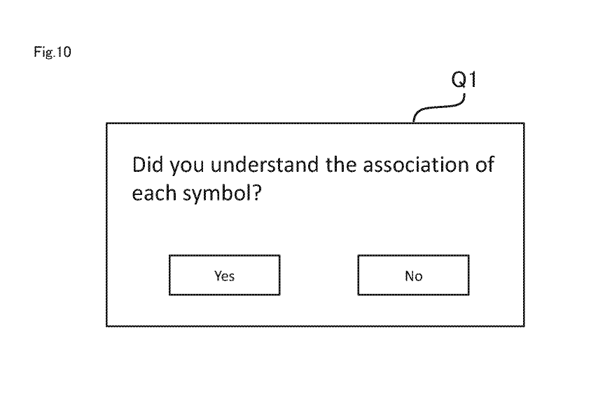

Thereafter, question screen Q1 is displayed to confirm whether the examinee has understood the association of each symbol (step S2-13). As shown in FIG. 10, the question screen Q1 includes “Yes” and “No” buttons. If the examinee selects “Yes” (“Yes” in step S2-14), step S2 shown in FIG. 6 is completed. If the examinee selects “No” (“No” in step S2-14), steps S2-1 to S2-13 are repeated.

A specific example of the procedure in step S2 in this embodiment is described above. In steps S2-2, S2-4, S2-6, S2-8, S2-10, and S2-12, the computer 100 may be configured such that sound is emitted from the audio output device 104 while displaying the phrases in speech balloons. Alternatively, the computer 100 may explain to the examinee the contents displayed in speech balloons by only sound from the audio output device 104 instead of displaying them in speech balloons. Alternatively, when the set 10 is drawn on a whiteboard, a paper medium, or the like, the robotic computer 100 may write by hand the concepts associated with each symbol. When the sixth and seventh symbols 6 and 7 are not included in the set 10, the association regarding the sixth and seventh symbols 6 and 7 is not performed.

Subsequently, in step S3 shown in FIG. 6, the computer 100 makes the examinee understand the recognition system represented by the set 10, based on the contrasting concepts between the second symbol 2 and the third symbol 3, between the fourth symbol 4 and the fifth symbol 5, and between the sixth symbol 6 and the seventh symbol 7.

The second symbol 2 and third symbol 3 have different shapes representing concepts contrasting with each other. For example, as shown in FIGS. 2 to 4, when the second symbol 2 has a “+” shape, then the third symbol 3 has a “−” shape, and as shown in FIG. 5, when the second symbol 2 has a “−” shape, then the third symbol 3 has a “+” shape.

The shapes of “+” and “−” have a relation of opposite polarities. Likewise, the examinee's own thought and the examinee's feeling from an object that produces conflict in the mind of the examinee have a relation of opposite polarities. For example, if the feeling from the object of the examinee's thought (hereinafter referred to as the “object”) is assumed to be “coldness in a boss at a workplace,” the feeling “coldness” is attributable to the examinee's thought, which is relatively “warm,” compared to the boss. However, the examinee is not usually aware of this fact. Thus, in view of the recognition system represented by the set 10, the examinee's own feeling (warmth) that is recognized by the examinee's thought and the feeling (coldness) from the object of the examinee's thought are respectively associated with the second symbol 2 and the third symbol 3, which represent contrasting concepts. This allows the examinee to easily understand that the examinee's thought and the examinee's feeling from the object have opposite concepts, and allows the causes of conflicts in the mind, i.e., unconsciousness and a blind spot, to be exposed in the examinee's thought.

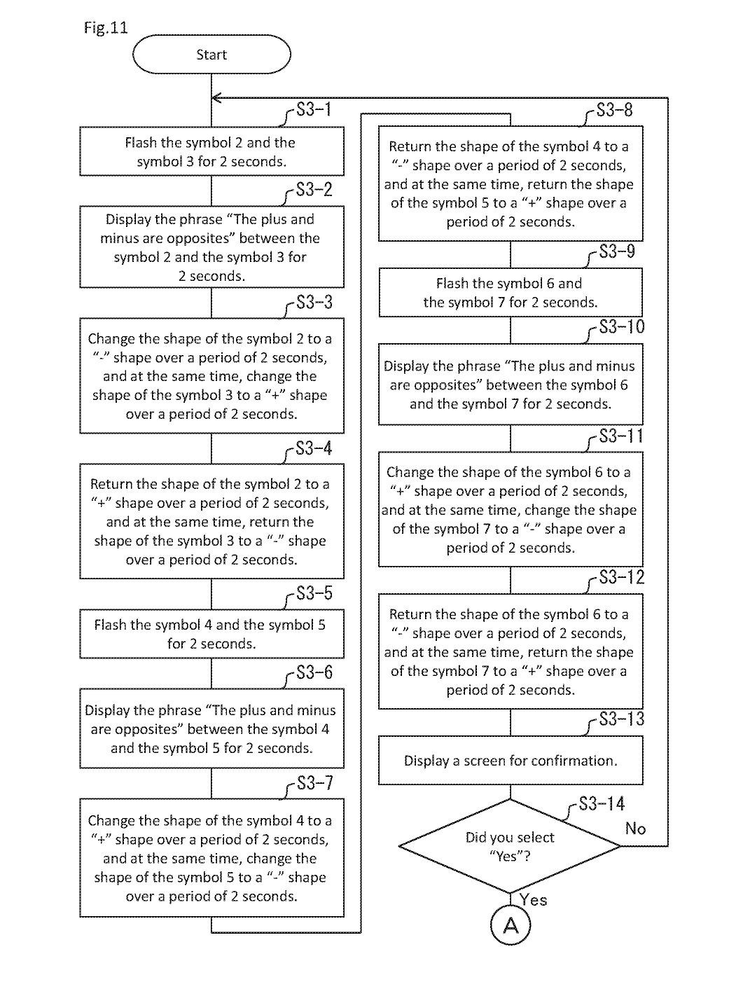

A specific example of the procedure in step S3 in this embodiment is described below. FIG. 11 is a flow chart illustrating a part of a specific procedure of step S3 shown in

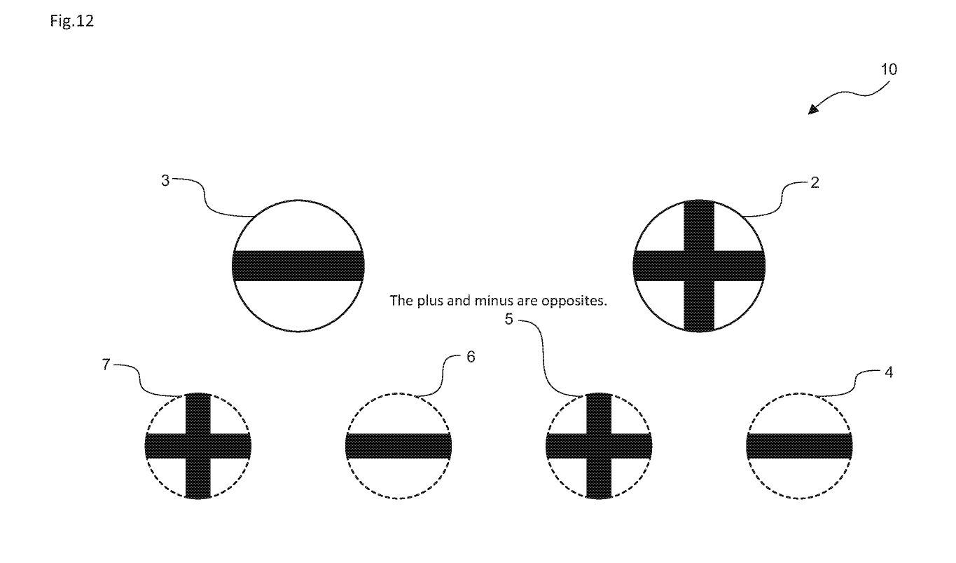

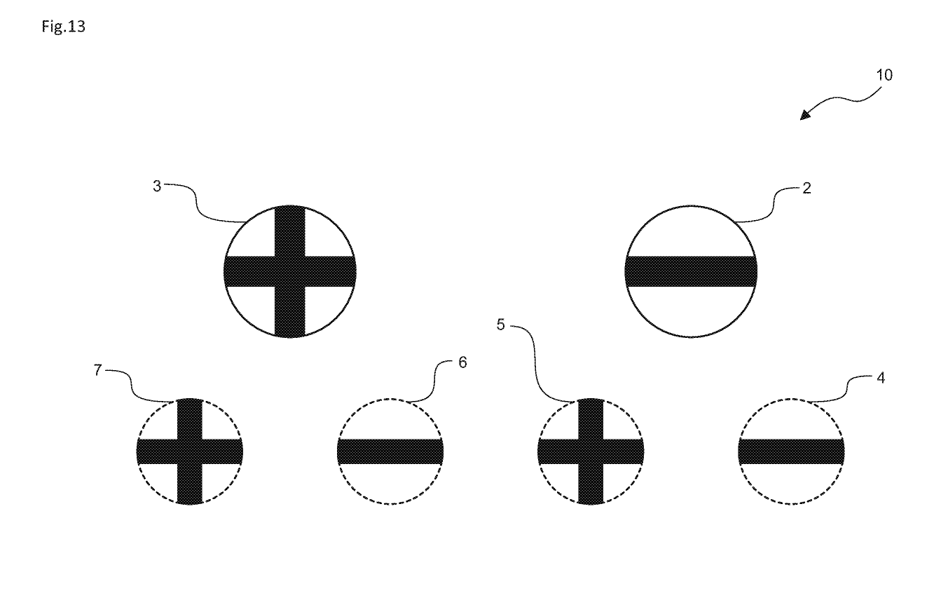

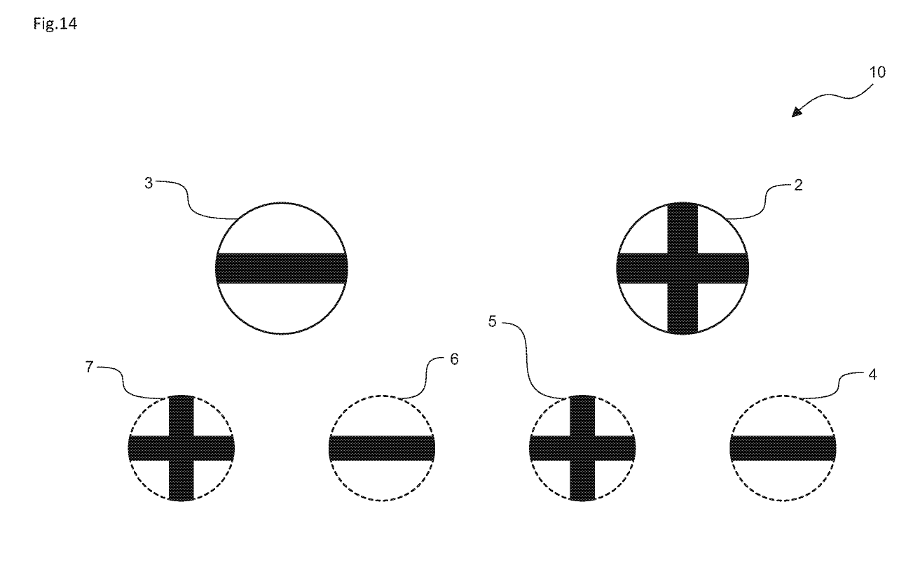

FIG. 6. First, the second symbol 2 and the third symbol 3 are flashed for 2 seconds in a state where the set 10 is displayed (step S3-1). Thereafter, as shown in FIG. 12, the phrase “The plus and minus are opposites” is displayed between the second symbol 2 and the third symbol 3 for 2 seconds (step S3-2). Then, as shown in FIG. 13, the shape of the second symbol 2 is changed to a “−” shape over a period of 2 seconds, and at the same time, the shape of the third symbol 3 is changed to a “+” shape over a period of 2 seconds (step S3-3). After that, as shown in FIG. 14, the shape of the second symbol 2 is returned to a “+” shape over a period of 2 seconds, and at the same time, the shape of the third symbol 3 is returned to a “−” shape over a period of 2 seconds (step S3-4).

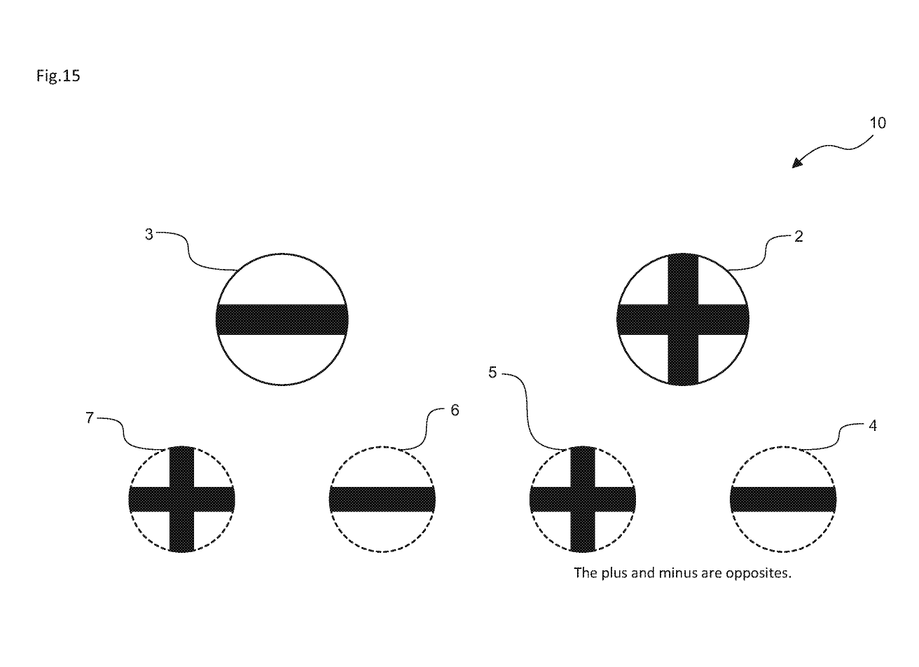

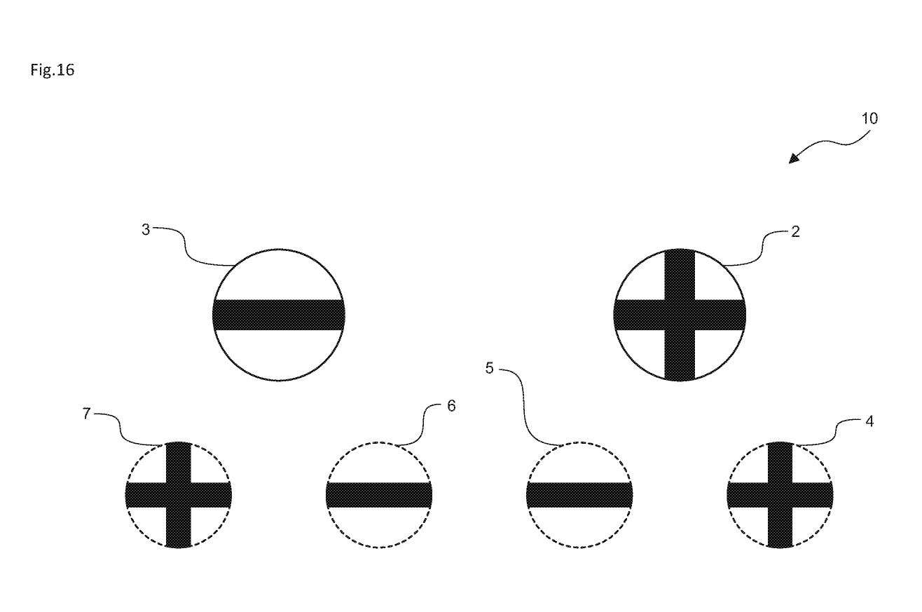

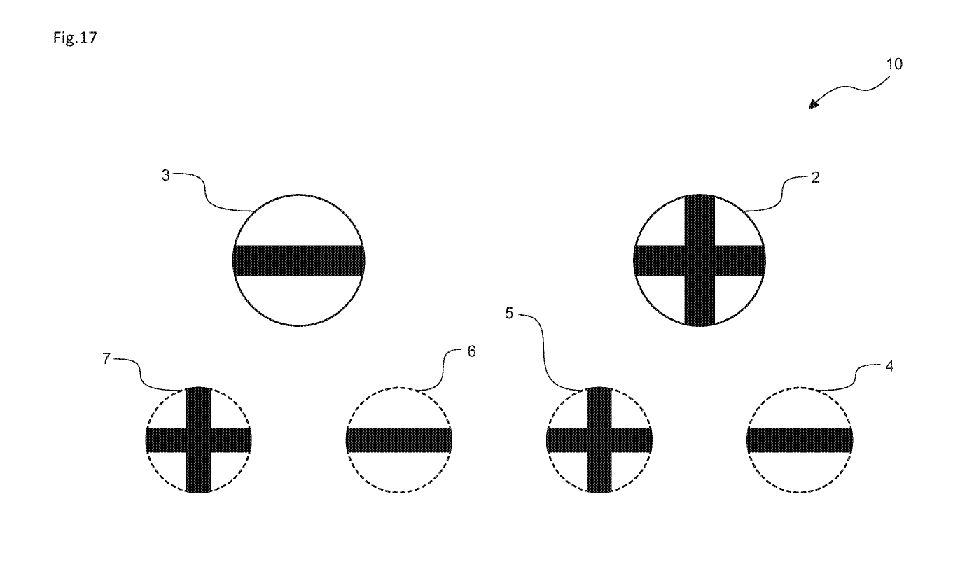

A similar process is performed for the fourth symbol 4 and the fifth symbol 5. Specifically, in step S3-5 in FIG. 11, the fourth symbol 4 and the fifth symbol 5 are flashed for 2 seconds, and then, as shown in FIG. 15, the phrase “The plus and minus are opposites” is displayed between the fourth symbol 4 and the fifth symbol 5 for 2 seconds (step S3-6). Thereafter, as shown in FIG. 16, the shape of the fourth symbol 4 is changed to a “+” shape over a period of 2 seconds, and at the same time, the shape of the fifth symbol 5 is changed to a “−” shape over a period of 2 seconds (step S3-7). Subsequently, as shown in FIG. 17, the shape of the fourth symbol 4 is returned to a “−” shape over a period of 2 seconds, and at the same time, the shape of the fifth symbol 5 is returned to a “+” shape over a period of 2 seconds (step S3-8).

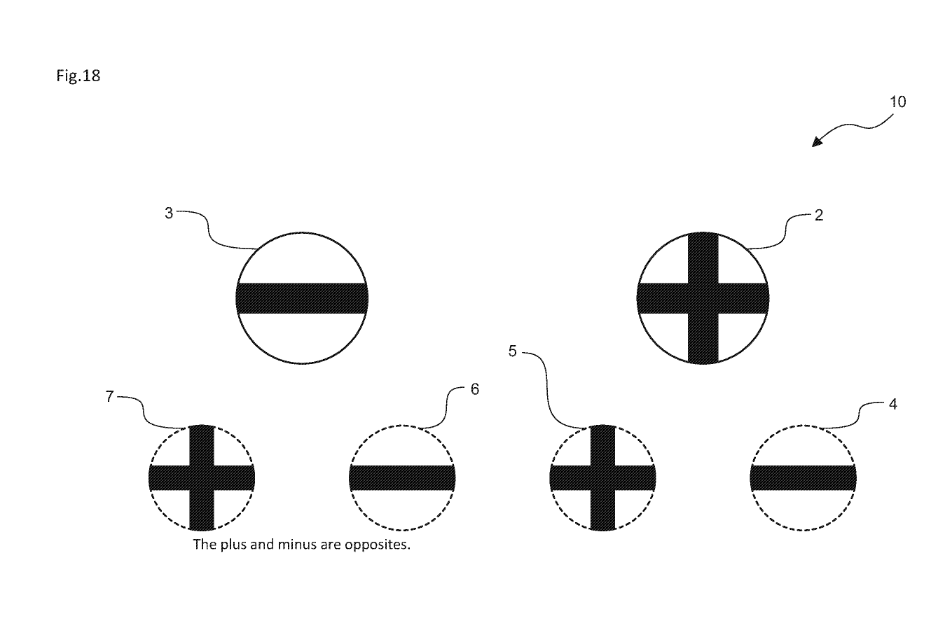

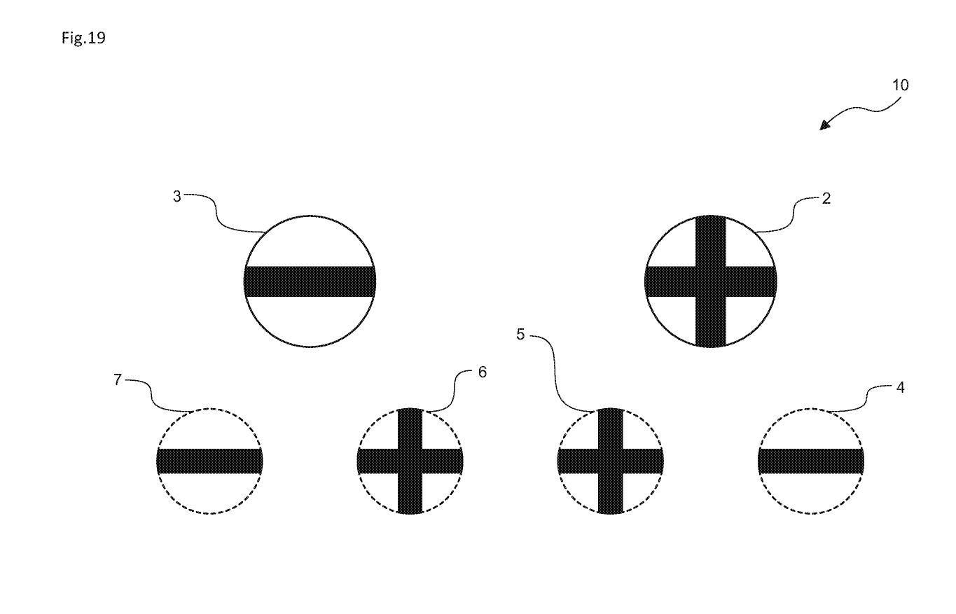

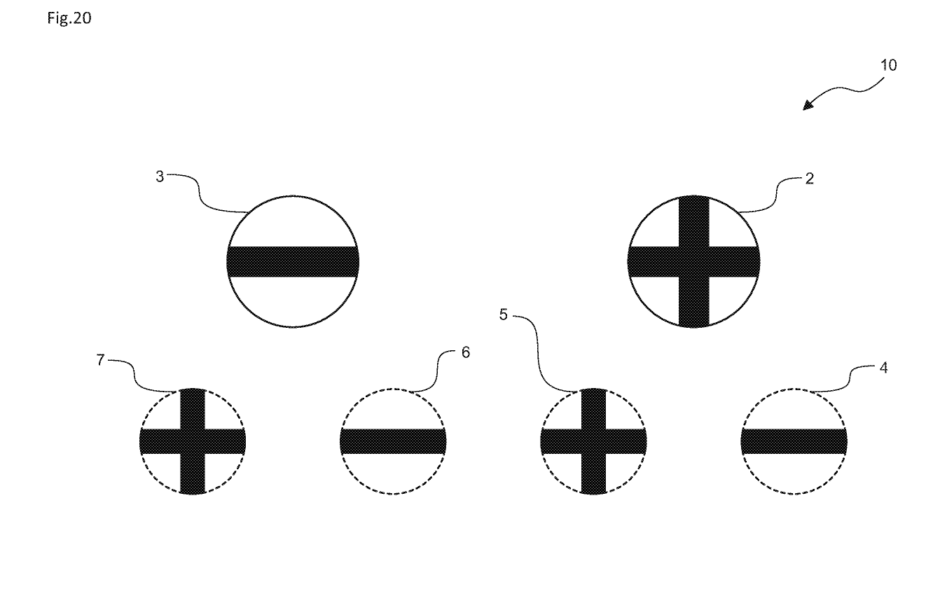

A similar process is performed for the sixth symbol 6 and the seventh symbol 7. Specifically, in step S3-9 in FIG. 11, the sixth symbol 6 and the seventh symbol 7 are flashed for 2 seconds, and then, as shown in FIG. 18, the phrase “The plus and minus are opposites” is displayed between the sixth symbol 6 and the seventh symbol 7 for 2 seconds (step S3-10). Thereafter, as shown in FIG. 19, the shape of the sixth symbol 6 is changed to a “+” shape over a period of 2 seconds, and at the same time, the shape of the seventh symbol 7 is changed to a “−” shape over a period of 2 seconds (step S3-11). Subsequently, as shown in FIG. 20, the shape of the sixth symbol 6 is returned to a “−” shape over a period of 2 seconds, and at the same time, the shape of the seventh symbol 7 is returned to a “+” shape over a period of 2 seconds (step S3-12). Steps S3-10 to S3-12 may be omitted.

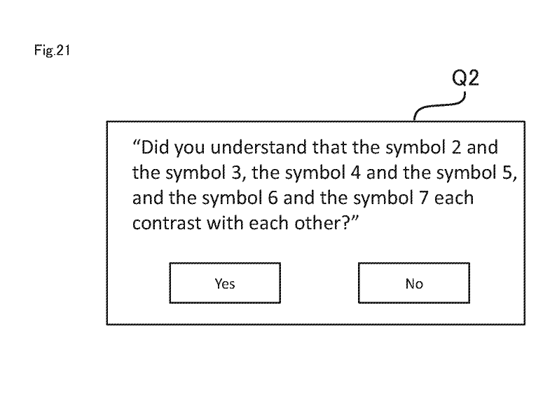

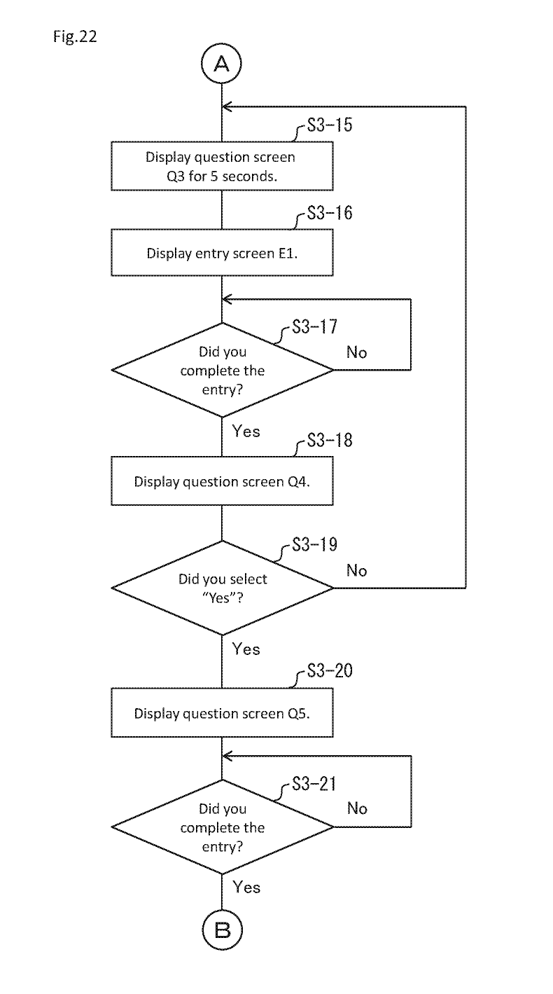

After that, question screen Q2 is displayed to confirm whether the examinee has understood the contrasting concepts between the symbols (step S3-13). As shown in FIG. 21, the question screen Q2 include the message “Did you understand that the symbol 2 and the symbol 3, the symbol 4 and the symbol 5, and the symbol 6 and the symbol 7 each contrast with each other?” and “Yes” and “No” buttons. If the examinee selects “Yes” (“Yes” in Step S3-14), the step proceeds to step S3-15 shown in FIG. 22. If the examinee selects “No” (“No” in step S3-14), steps S3-1 to S3-13 are repeated.

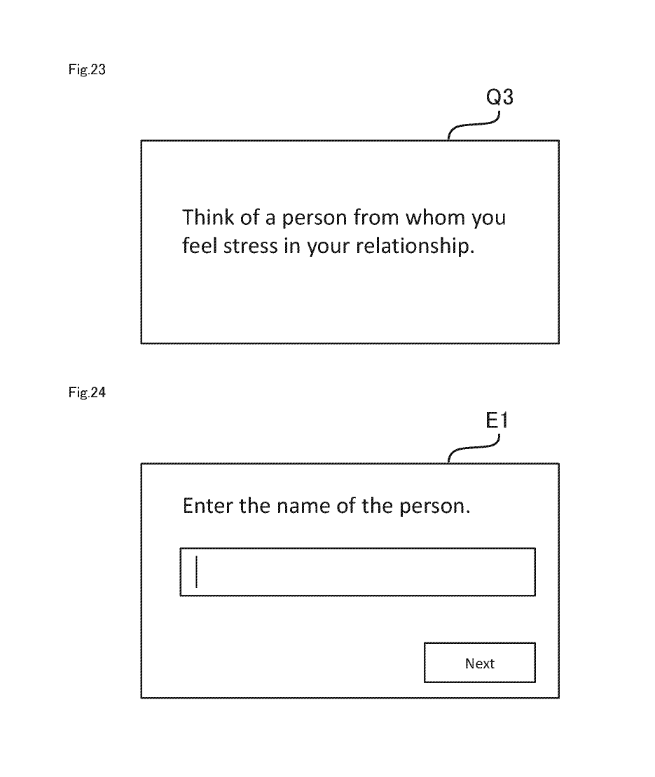

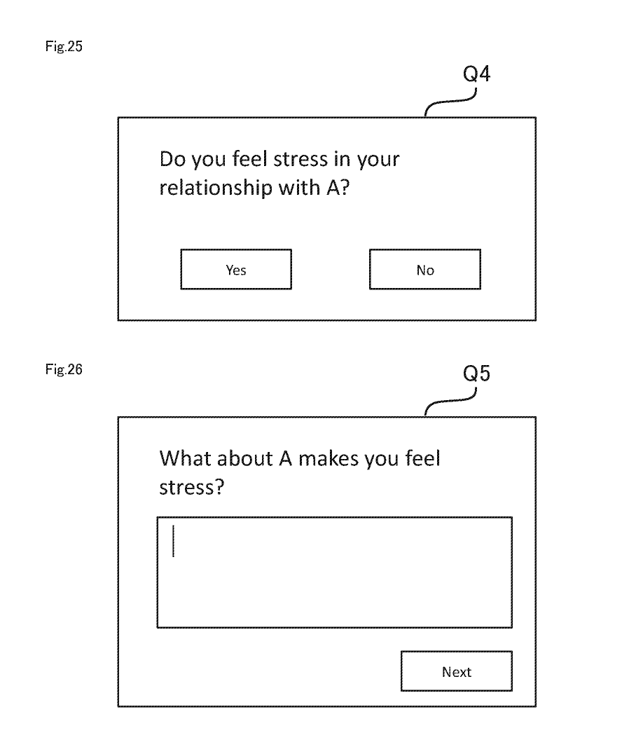

Subsequently, in step S3-15 shown in FIG. 22, question screen Q3 shown in FIG. 23 is displayed for 5 seconds. The question screen Q3 includes the message “Think of a person from whom you feel stress in your relationship.” Thereafter, entry screen E1 shown in FIG. 24 is displayed (step S3-16). The entry screen E1 includes the message “Enter the name of the person,” an entry field, and a “Next” button. When the examinee enters the name of the person in the entry field and selects the “Next” button, the entry is completed. In this embodiment, the name of the person is assumed to be “A.” After the examinee completes the entry on the entry screen E1 (“Yes” in step S3-17), question screen Q4 shown in FIG. 25 is displayed (step S3-18). The question screen Q4 includes the message “Do you feel stress in your relationship with A?” and “Yes” and “No” buttons. If the examinee selects “Yes” on the question screen Q4 (“Yes” in step S3-19), question screen Q5 shown in FIG. 26 is displayed (step S3-20). The question screen Q5 includes the message “What about A makes you feel stress?”, an entry field, and a “Next” button. When the examinee enters an answer in the entry field and selects the “Next” button, the entry is completed. In this embodiment, the term “coldness” is assumed to be entered as the answer.

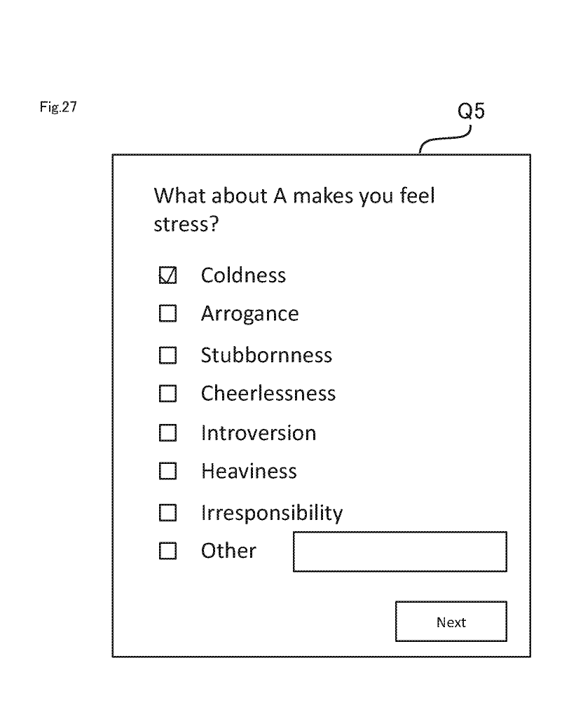

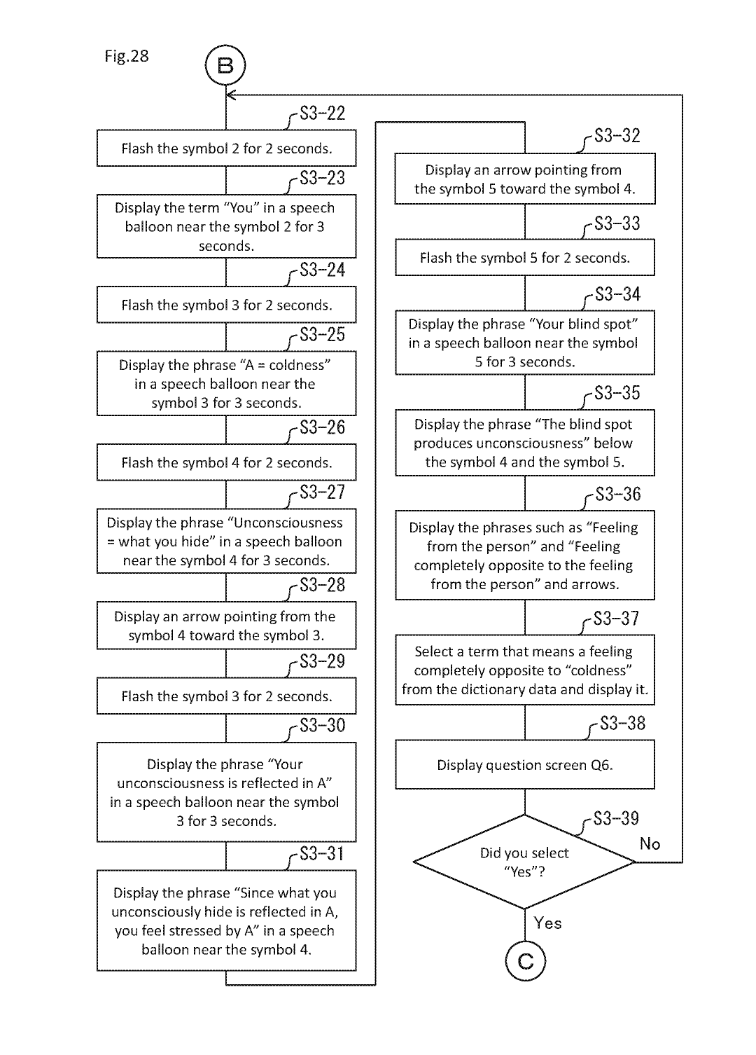

The question screen Q5 shown in FIG. 26 is in a form in which the examinee freely enters text in the entry field, but may be in a form in which the examinee chooses an answer from multiple choices as shown in a modification shown in FIG. 27. After the examinee completes the entry on the question screen Q5 (“Yes” in step S3-21), the step proceeds to step S3-22 shown in FIG. 28.

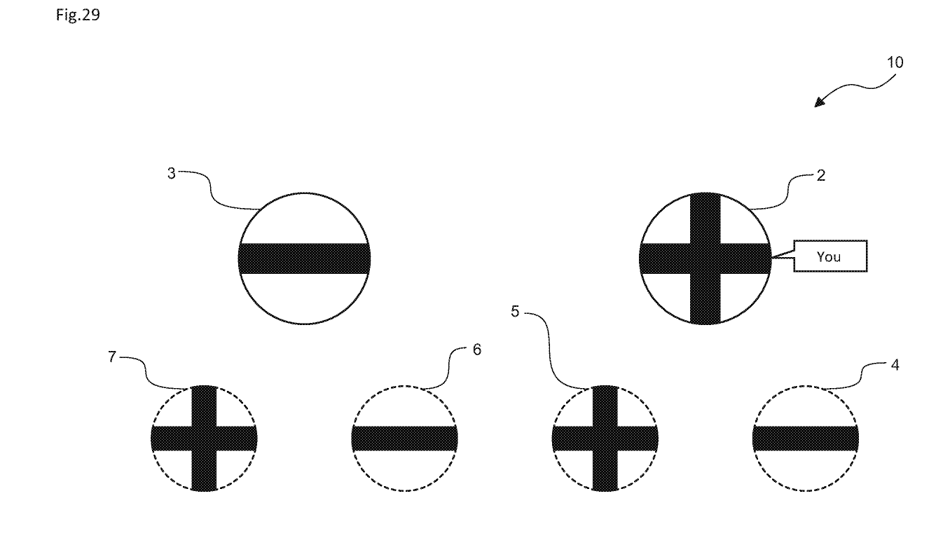

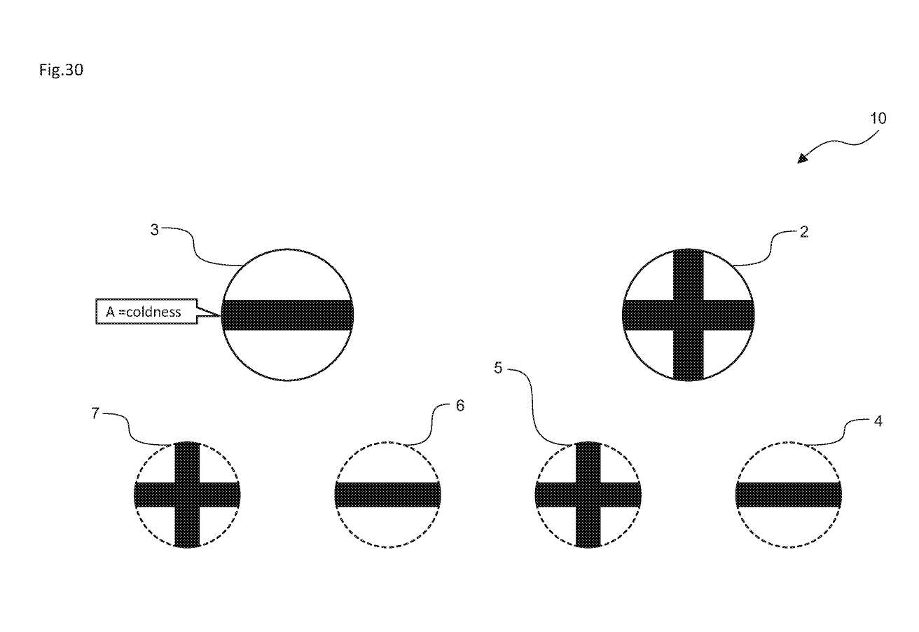

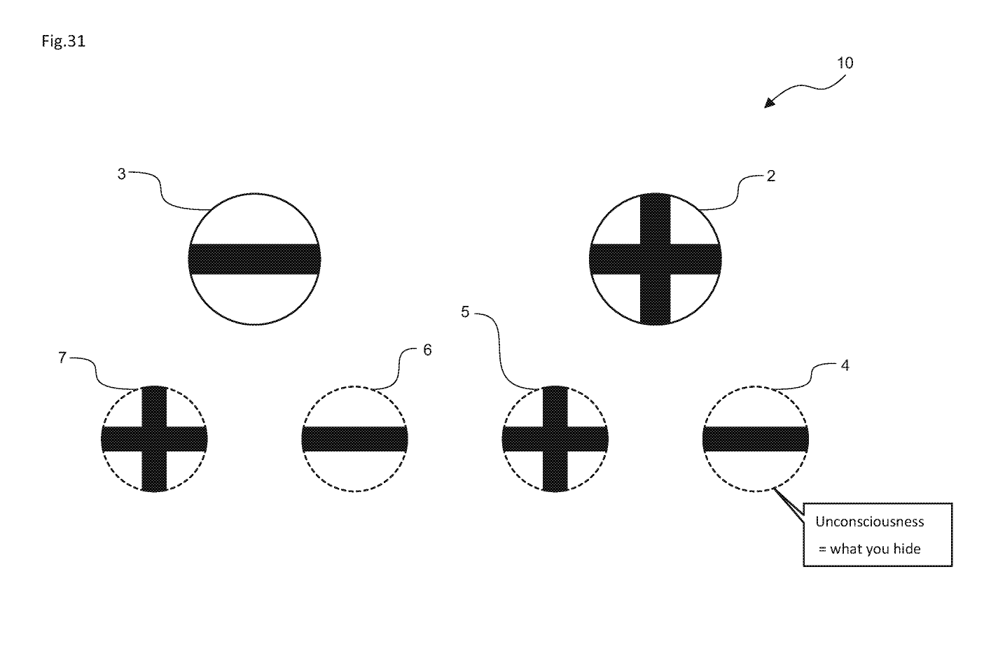

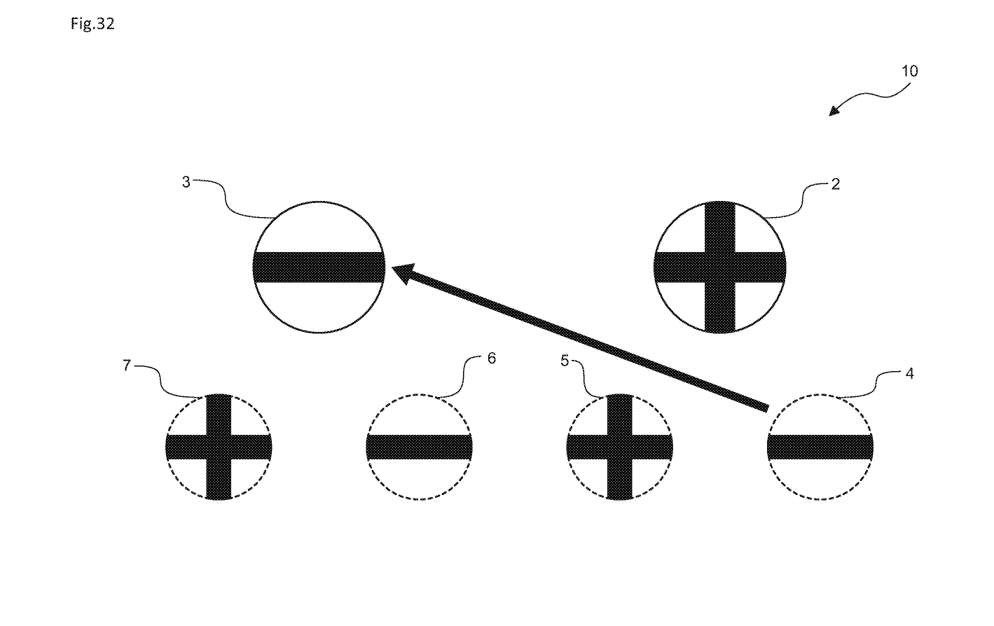

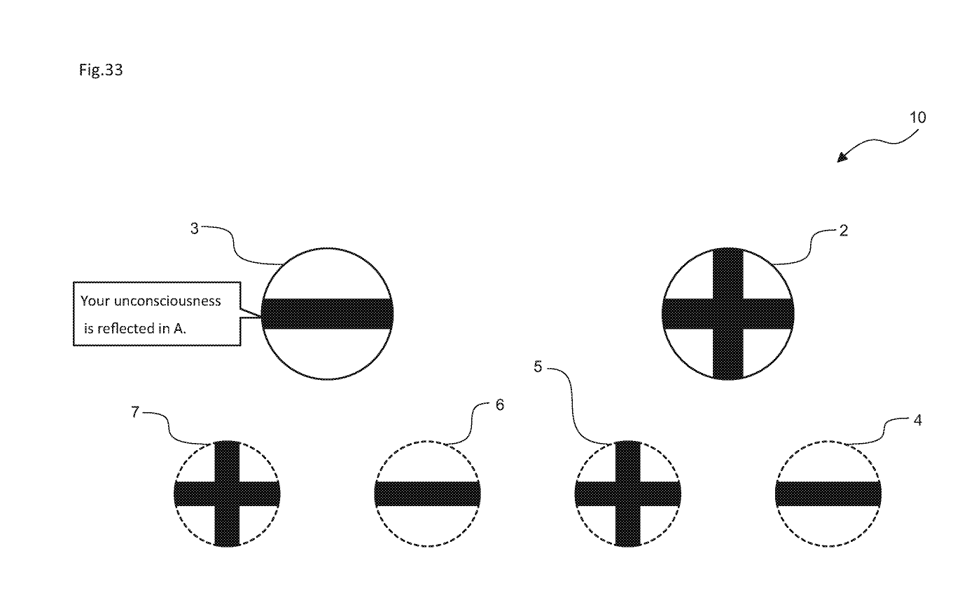

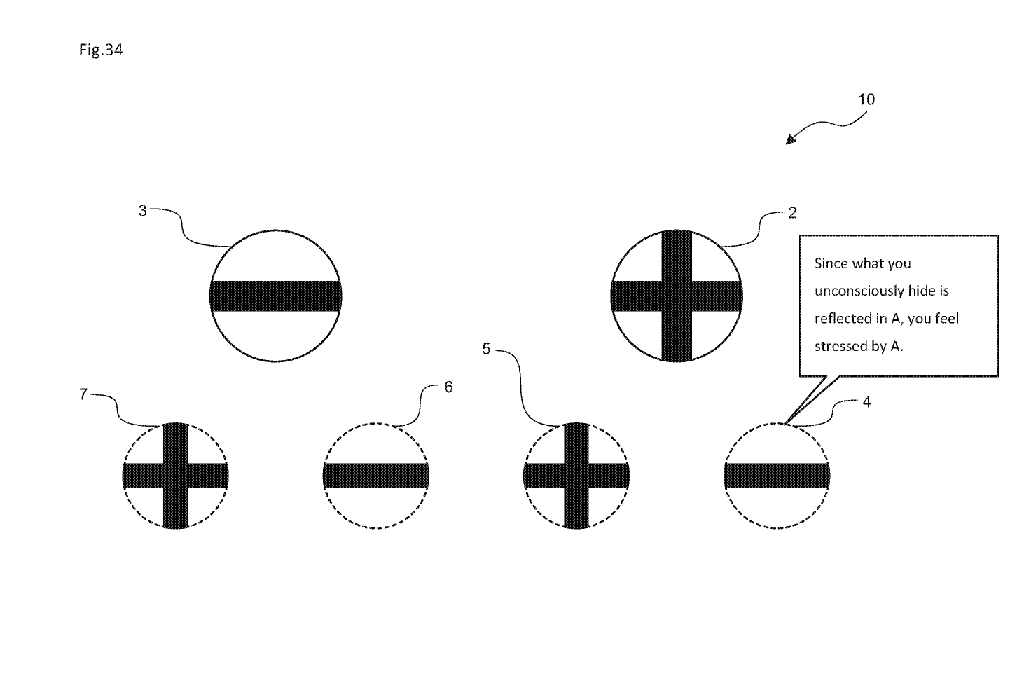

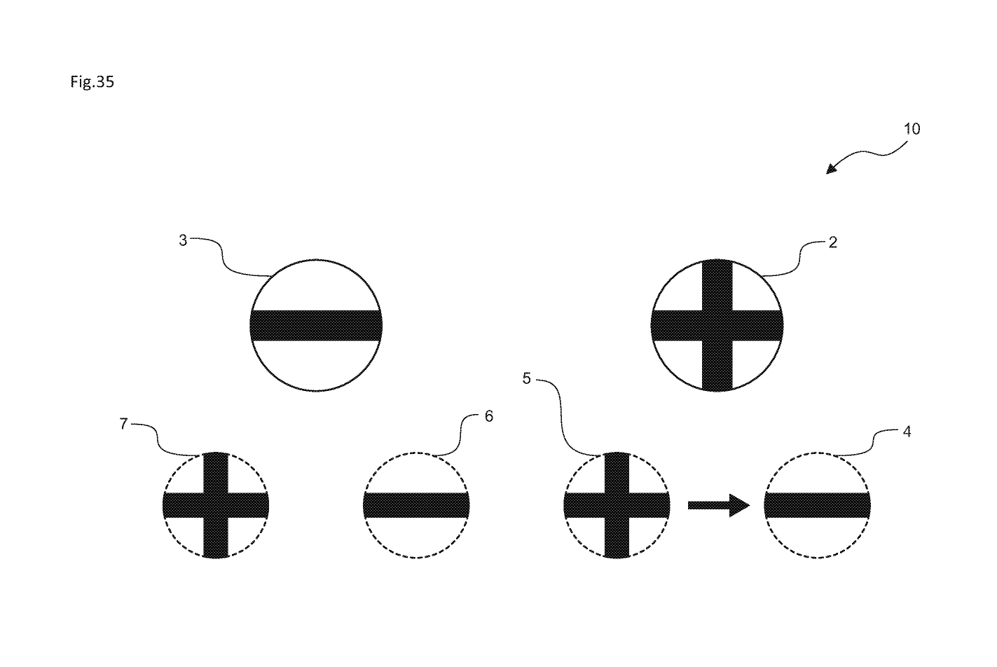

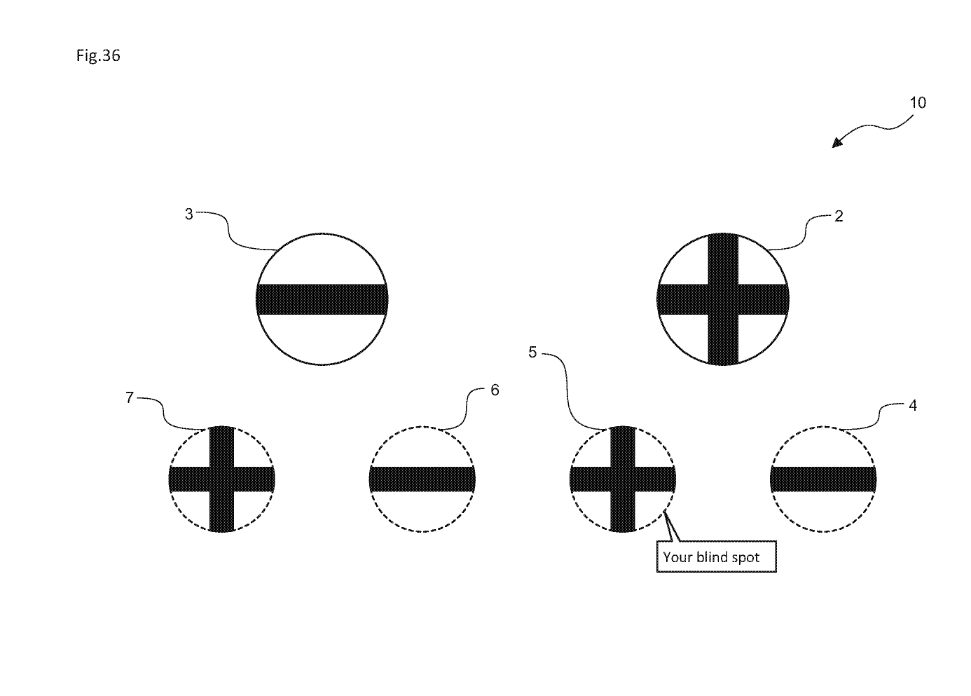

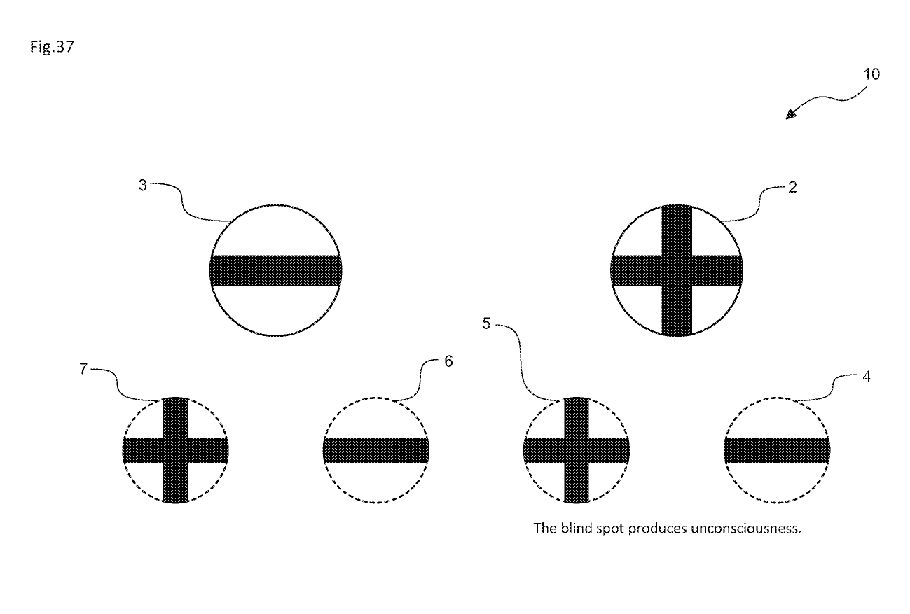

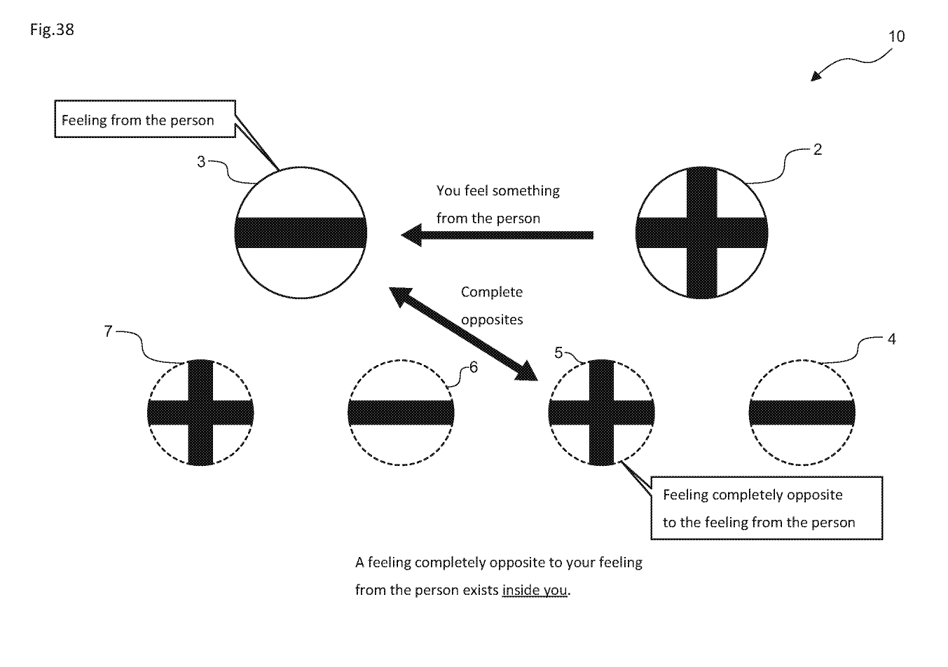

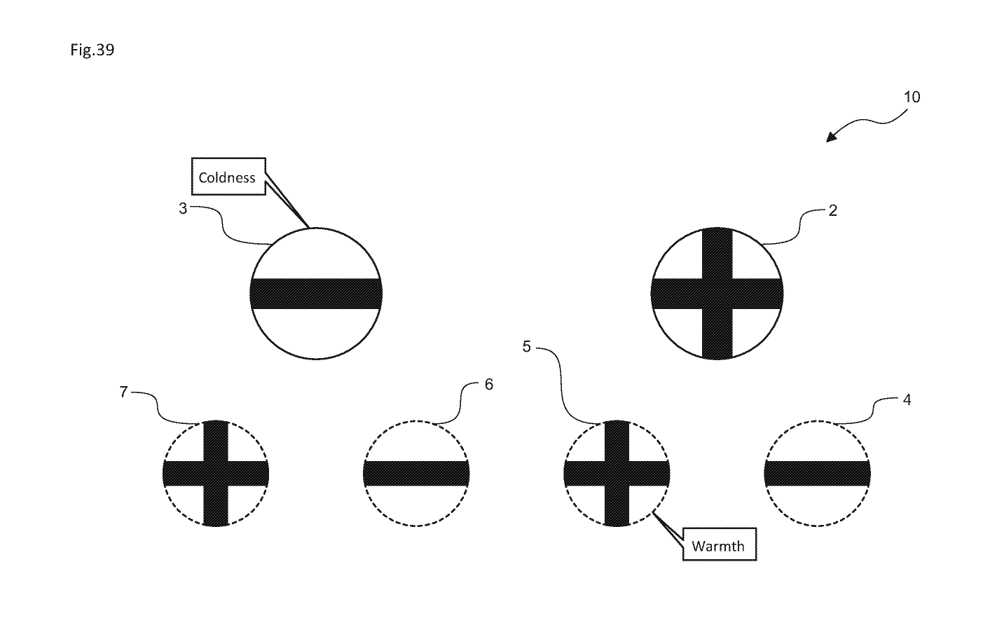

In step S3-22, the screen is switched to the screen of the set 10, and the second symbol 2 is flashed for 2 seconds. Thereafter, as shown in FIG. 29, the term “You” is displayed in a speech balloon near the second symbol 2 (step S3-23). Furthermore, the third symbol 3 is flashed for 2 seconds (step S3-24), and then, as shown in FIG. 30, the phrase “A =coldness” is displayed in a speech balloon near the third symbol 3 for 3 seconds (step S3-25). Furthermore, the fourth symbol 4 is flashed for 2 seconds (step S3-26), and then, as shown in FIG. 31, the phrase “Unconsciousness =what you hide” is displayed in a speech balloon near the fourth symbol 4 for 3 seconds (step S3-27). Thereafter, as shown in FIG. 32, an arrow is displayed pointing from the fourth symbol 4 toward the third symbol 3 (step S3-28). Furthermore, the third symbol 3 is flashed for 2 seconds (step S3-29), and then, as shown in FIG. 33, the phrase “Your unconsciousness is reflected in A” is displayed in a speech balloon near the third symbol 3 (step S3-30). Subsequently, as shown in FIG. 34, the phrase “Since what you unconsciously hide is reflected in A, you feel stressed by A” is displayed in a speech balloon near the fourth symbol 4 (step S3-31). Thereafter, as shown in FIG. 35, an arrow is displayed pointing from the fifth symbol 5 toward the fourth symbol 4 (step S3-32). Furthermore, the fifth symbol 5 is flashed for 2 seconds (step S3-33), and then, as shown in FIG. 36, the phrase “Your blind spot” is displayed in a speech balloon near the fifth symbol 5 (step S3-34). Subsequently, as shown in FIG. 37, the phrase “The blind spot produces unconsciousness” is displayed below the fourth symbol 4 and the fifth symbol 5 (step S3-35). Furthermore, as shown in FIG. 38, an arrow is displayed pointing from the second symbol 2 toward the third symbol 3, and at the same time, the phrase “You feel something from the person” is displayed above the arrow; then, the phrase “Feeling from the person” is displayed in a speech balloon on the upper-left side of the third symbol 3; subsequently, between the third symbol 3 and the fifth symbol 5, an arrow indicating that they are opposites is displayed, and at the same time, the phrase “Complete opposites” is displayed above the arrow; further, the phrase “Feeling completely opposite to the feeling from the person” is displayed in a speech balloon on the lower-right side of the fifth symbol 5, and finally, the phrase “A feeling completely opposite to your feeling from the person exists inside you” is displayed below the set 10 (step S3-36). The arrow pointing from the second symbol 2 toward the third symbol 3 is preferably green. Subsequently, the dictionary data 113 shown in FIG. 1 is read, and a term that means a feeling completely opposite to “coldness” (“warmth”) is selected from the dictionary data 113; as shown in FIG. 39, the term “Coldness” is displayed near the third symbol 3, and the term “Warmth” is also displayed in a speech balloon near the fifth symbol 5 (step S3-37). Table 1 shows an example of the dictionary data 113.

TABLE 1

| | |
|---|---|
| Warmth | Coldness |
| Modesty | Arrogance |
| Compliant | Stubbornness |
| Cheerfulness | Cheerlessness |
| Extroversion | Introversion |
| Lightness | Heaviness |
| Strong sense of responsibility | Irresponsibility |
| . . . | . . . |

When the term entered by the user in the question screen Q5 (FIG. 26 or FIG. 27) is not included in the dictionary data 113, the computer 100 may access any dictionary data on a cloud and obtain a term completely opposite to the entered term. Furthermore, each time a term that is not included in the dictionary data 113 is entered, the dictionary data 113 may be updated through machine learning.

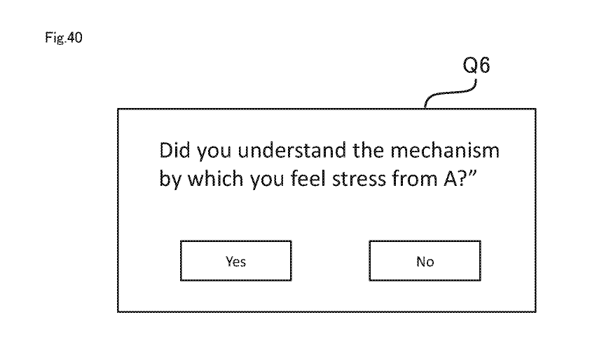

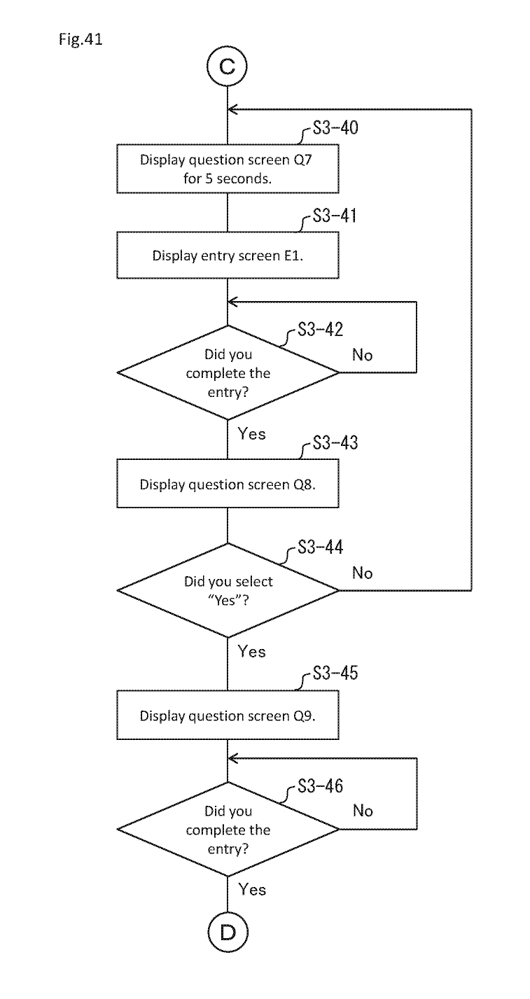

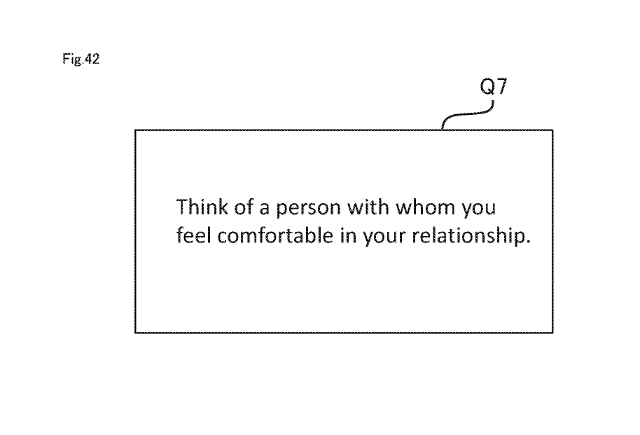

After step S3-37, question screen Q6 shown in FIG. 40 is displayed (step S3-38). The question screen Q6 includes the message “Did you understand the mechanism by which you feel stress from A?” and “Yes” and “No” buttons. If the examinee selects “Yes” (“Yes” in step S3-39), the step proceeds to step S3-40 shown in FIG. 41. If the examinee selects “No” (“No” in step S3-39), steps S3-22 to S3-38 are repeated.

Steps S3-15 to S3-21 shown in FIG. 22 and steps S3-22 to S3-39 shown in FIG. 28 may be omitted. Specifically, if “Yes” is selected in step S3-14 shown in FIG. 11, the step may proceed to step S3-40 shown in FIG. 41.

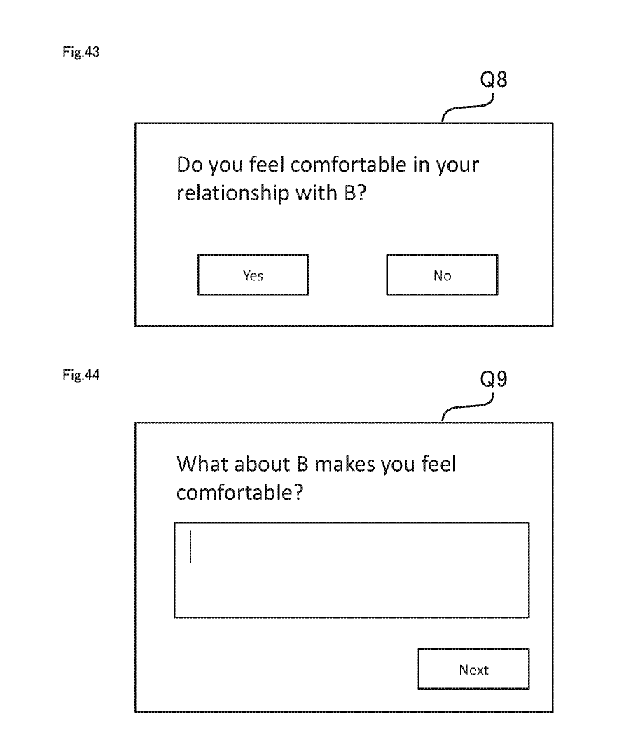

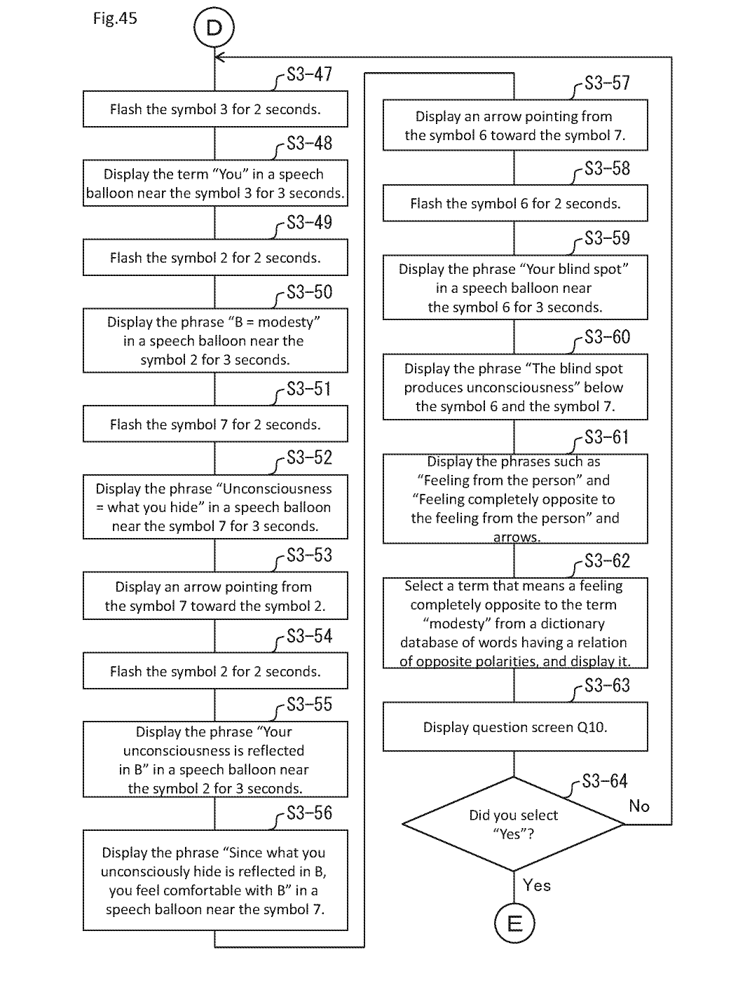

Subsequently, in step S3-40 shown in FIG. 41, question screen Q7 shown in FIG. 42 is displayed for 5 seconds. The question screen Q7 includes the message “Think of a person with whom you feel comfortable in your relationship.” Thereafter, the entry screen E1 shown in FIG. 24 is displayed (step S3-41). The entry screen E1 includes the message “Enter the name of the person,” an entry field, and a “Next” button. When the examinee enters the name of the person in the entry field and selects the “Next” button, the entry is completed. In this embodiment, the name of the person is assumed to be “B.” After the examinee completes the entry on the entry screen E1 (“Yes” in step S3-42), question screen Q8 shown in FIG. 43 is displayed (step S3-43). The question screen Q8 includes the message “Do you feel comfortable in your relationship with B?” and “Yes” and “No” buttons. If the examinee selects “Yes” on the question screen Q8 (“Yes” in step S3-44), question screen Q9 shown in FIG. 44 is displayed (step S3-45). The question screen Q9 includes the message “What about B makes you feel comfortable?”, an entry field, and a “Next” button. When the examinee enters an answer in the entry field and select the “Next” button, the entry is completed. In this embodiment, the term “modesty” is assumed to be entered as the answer. After the examinee completes the entry on the question screen Q9 (Yes in step S3-46), the step proceeds to step S3-47 shown in FIG. 45.

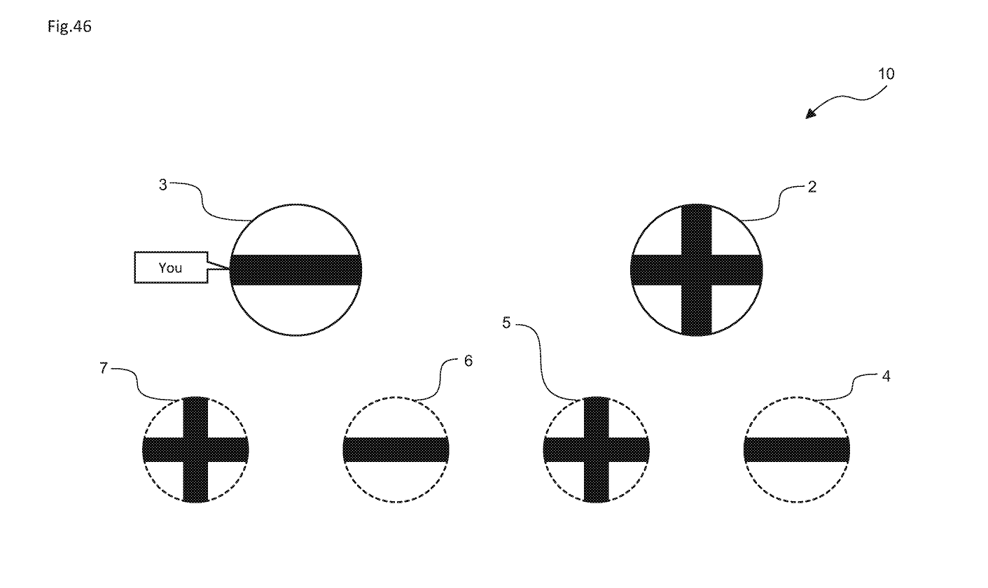

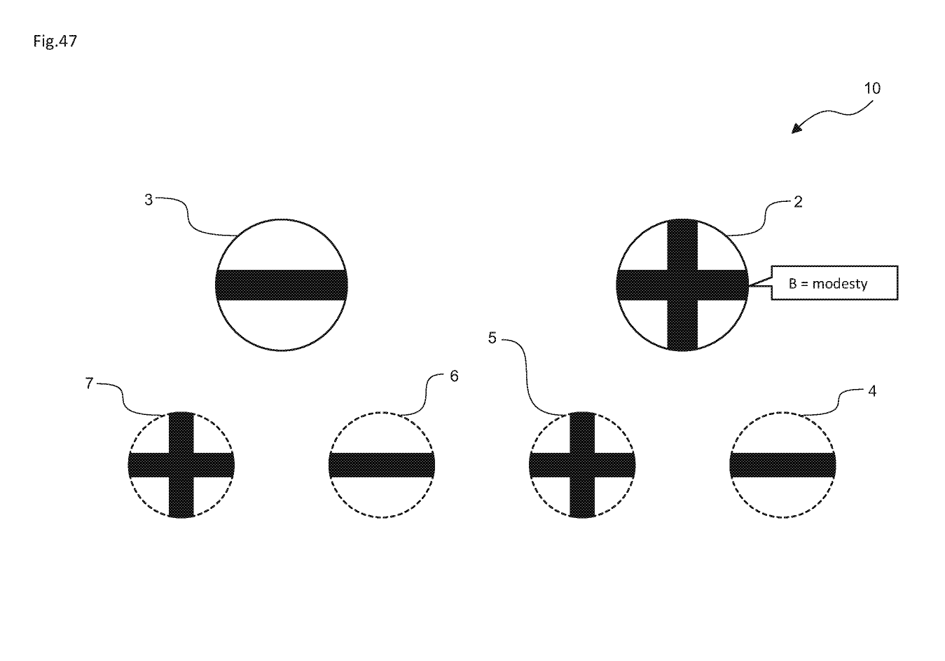

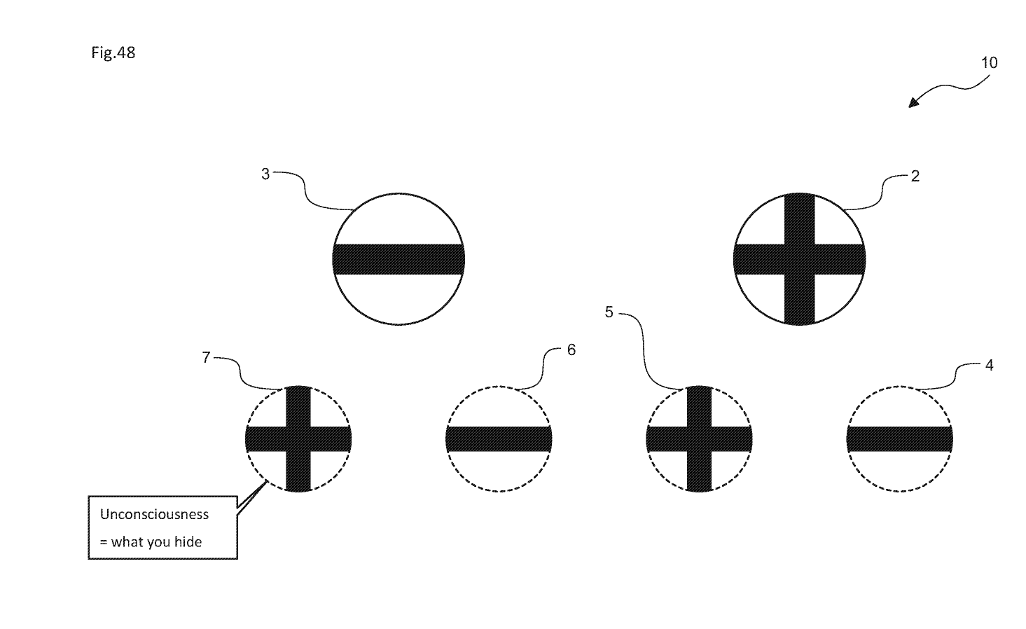

In step S3-47, the screen is switched to the screen of the set 10, and the third symbol 3 is flashed for 2 seconds. Thereafter, as shown in FIG. 46, the term “You” is displayed in a speech balloon near the third symbol 3 (step S3-48). Further, the second symbol 2 is flashed for 2 seconds (step S3-49), and then, as shown in FIG. 47, the phrase “B =modesty” is displayed in a speech balloon near the second symbol 2 for 3 seconds (step S3-50). Furthermore, the seventh symbol 7 is flashed for 2 seconds (step S3-51), and then, as shown in FIG. 48, the phrase

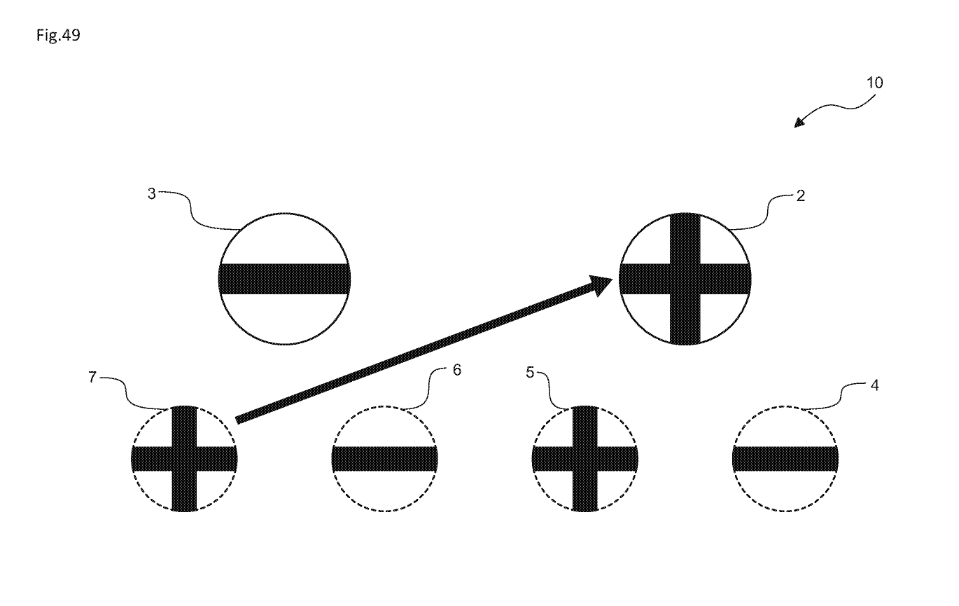

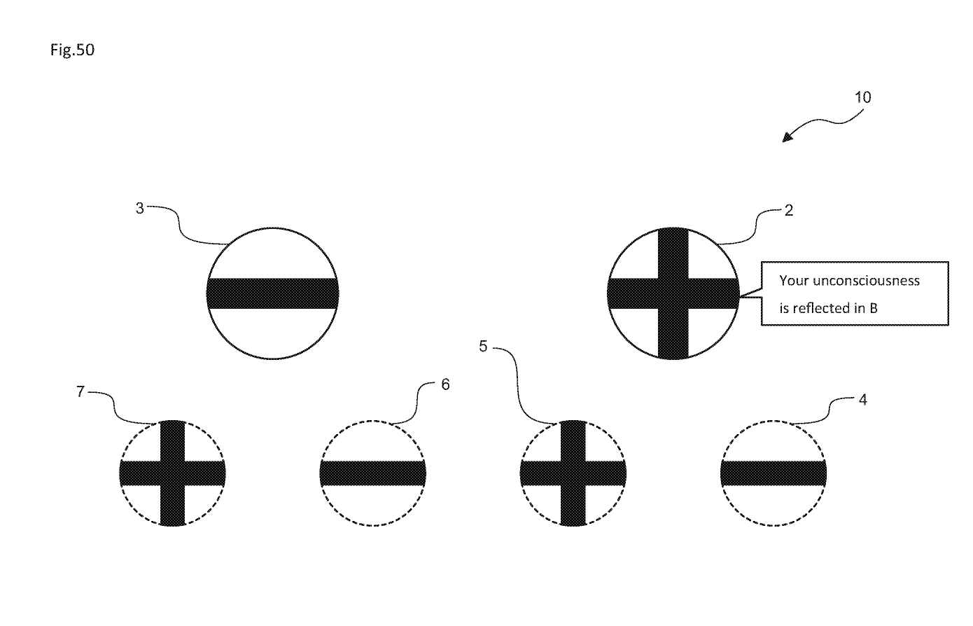

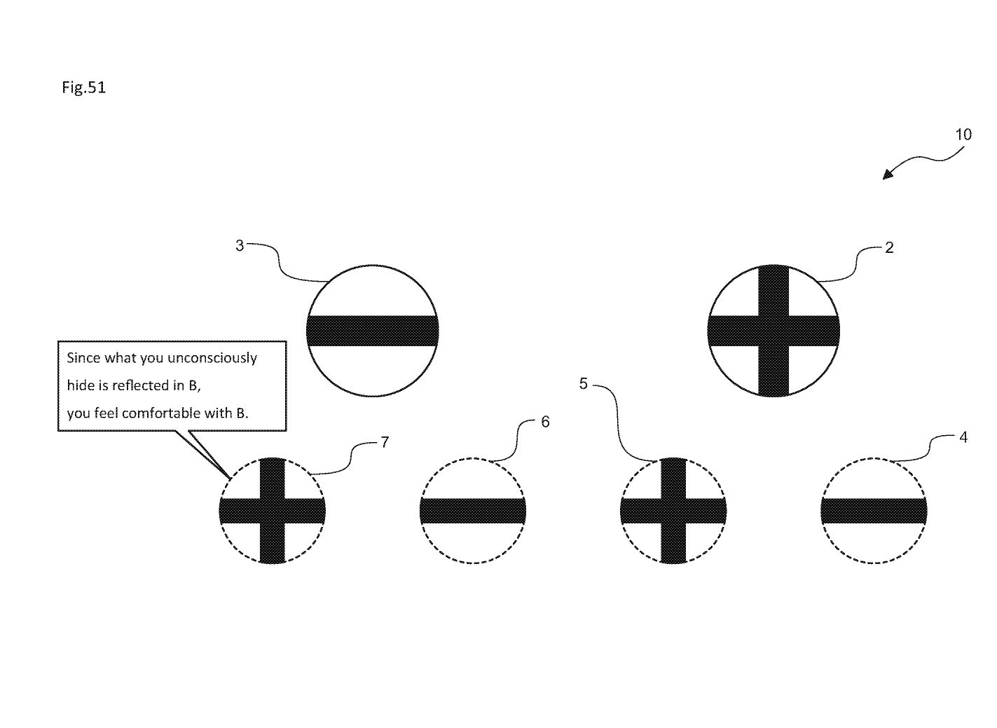

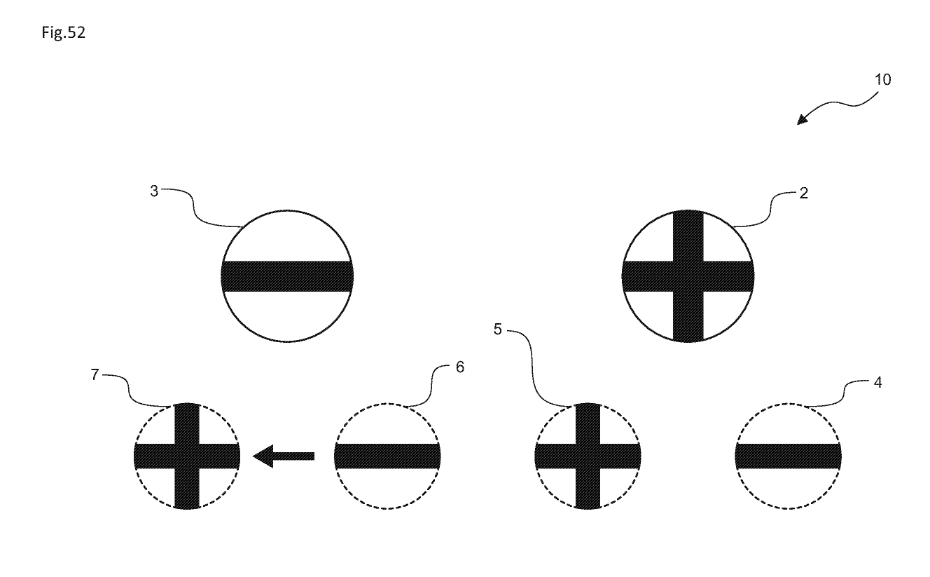

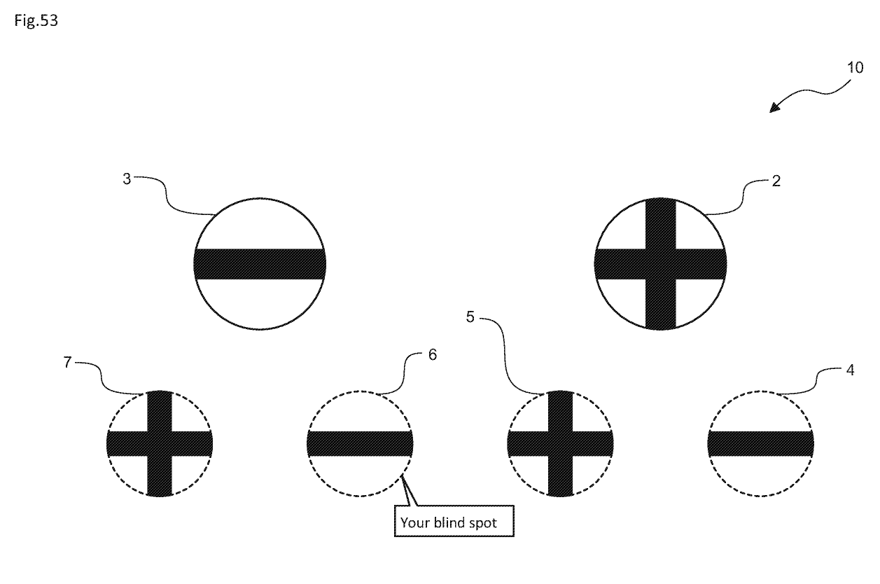

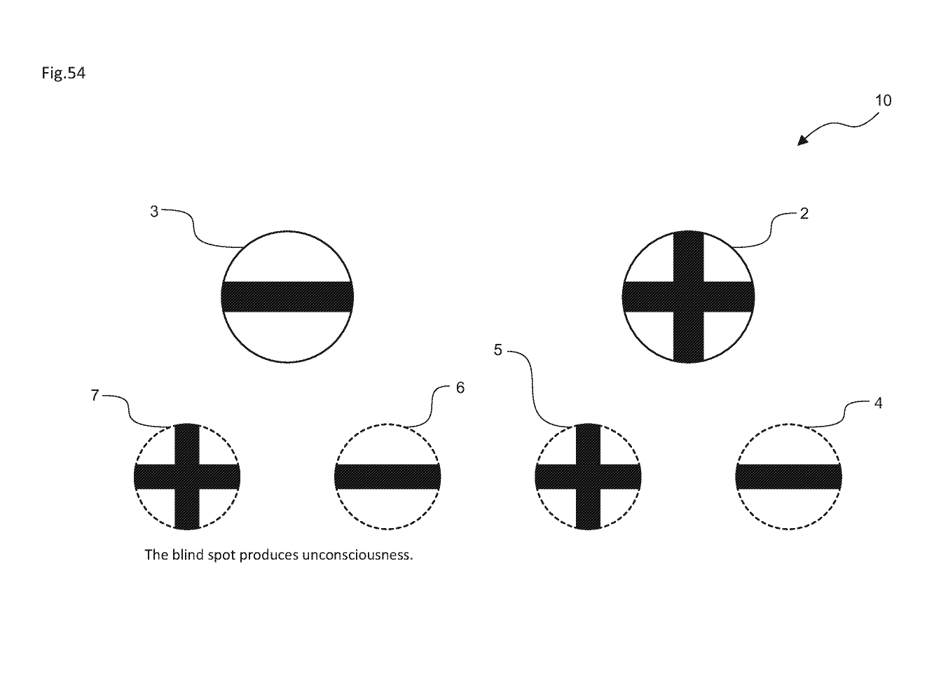

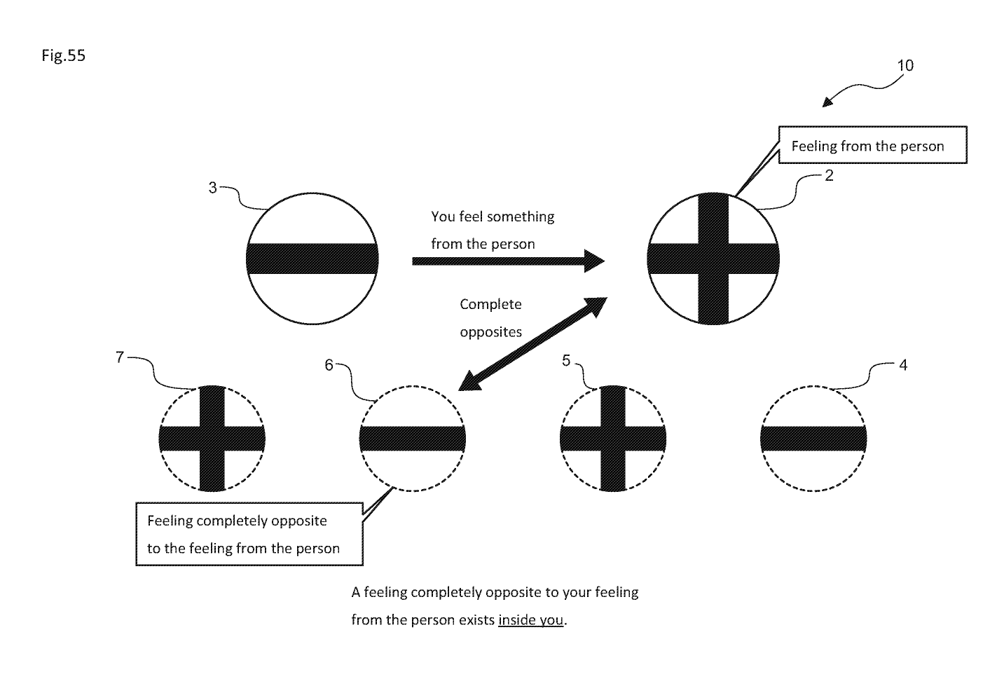

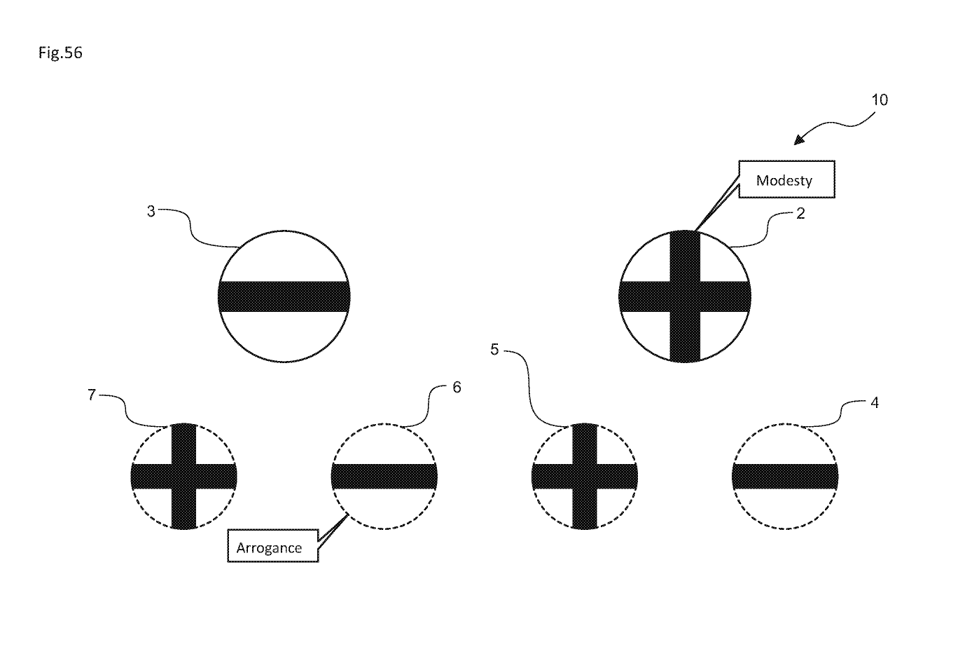

“Unconsciousness =what you hide” is displayed in a speech balloon near the seventh symbol 7 for 3 seconds (step S3-52). Thereafter, as shown in FIG. 49, an arrow is displayed pointing from the seventh symbol 7 toward the second symbol 2 (step S3-53). Further, the second symbol 2 is flashed for 2 seconds (step S3-54), and then, as shown in FIG. 50, the phrase “Your unconsciousness is reflected in B” is displayed in a speech balloon near the second symbol 2 (step S3-55). Subsequently, as shown in FIG. 51, the phrase “Since what you unconsciously hide is reflected in B, you feel comfortable with B” is displayed in a speech balloon near the seventh symbol 7 (step S3-56). Thereafter, as shown in FIG. 52, an arrow is displayed pointing from the sixth symbol 6 toward the seventh symbol 7 (step S3-57). Further, the sixth symbol 6 is flashed for 2 seconds (step S3-58), and then, as shown in FIG. 53, the phrase “Your blind spot” is displayed in a speech balloon near the sixth symbol 6 (step S3-59). Subsequently, as shown in FIG. 54, the phrase “The blind spot produces unconsciousness” is displayed below the sixth symbol 6 and the seventh symbol 7 (step S3-60). Further, as shown in FIG. 55, an arrow is displayed pointing from the third symbol 3 toward the second symbol 2, and at the same time, the phrase “You feel something from the person” is displayed above the arrow; then, the phrase “Feeling from the person” is displayed in a speech balloon on the upper-right side of the second symbol 2; subsequently, between the second symbol 2 and the sixth symbol 6, an arrow indicating that they are opposites is displayed, and at the same time, the phrase “Complete opposites” is displayed above the arrow; furthermore, the phrase “Feeling completely opposite to the feeling from the person” is displayed in a speech balloon on the lower-left side of the sixth symbol 6, and finally, the phrase “A feeling completely opposite to your feeling from the person exists inside you” is displayed below the set 10 (step S3-61). The arrow pointing from the third symbol 3 toward the second symbol 2 is preferably green. Subsequently, the dictionary data 113 shown in FIG. 1 is read, and a term that means a feeling completely opposite to the term “modesty” (“arrogance”) is selected from the dictionary data 113; as shown in FIG. 56, the term “Modesty” is displayed near the second symbol 2, and the term “Arrogance” is also displayed in a speech balloon near the sixth symbol 6 (step S3-62).

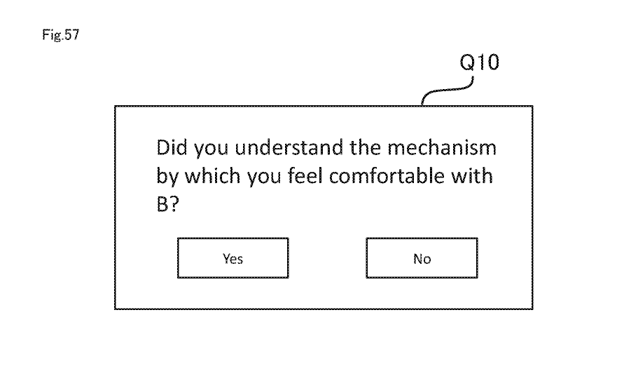

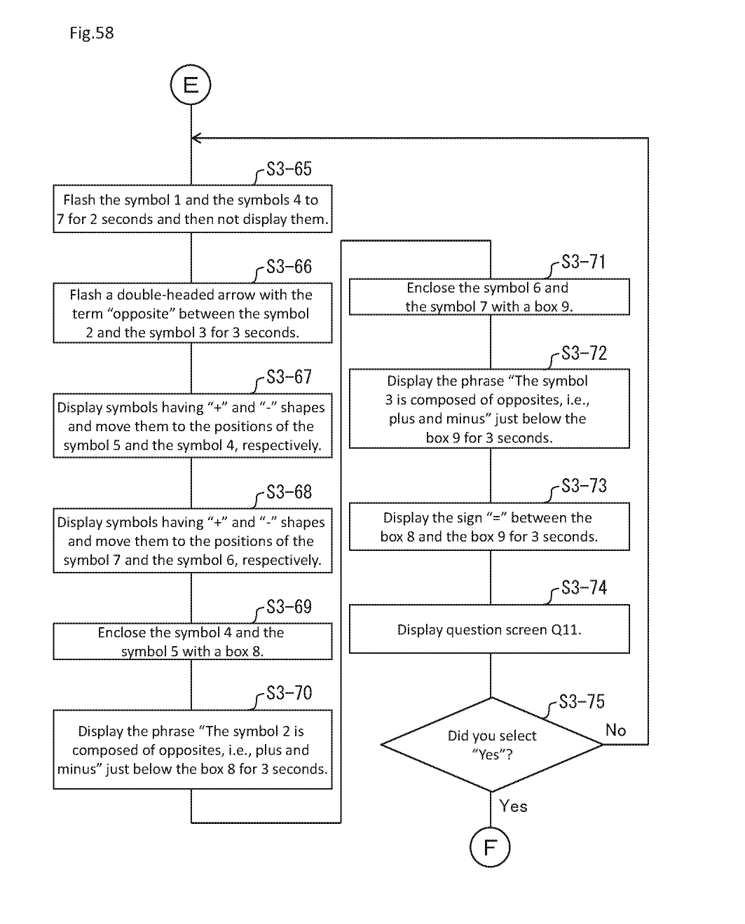

Thereafter, question screen Q10 shown in FIG. 57 is displayed (step S3-63). The question screen Q10 includes the message “Did you understand the mechanism by which you feel comfortable with B?” and “Yes” and “No” buttons. If the examinee selects “Yes” (“Yes” in step S3-64), the step proceeds to step S3-65 shown in FIG. 58. If the examinee selects “No” (“No” in step S3-64), steps S3-47 to S3-63 are repeated.

Steps S3-40 to S3-46 shown in FIG. 41 and steps S3-47 to S3-64 shown in FIG. 45 may be omitted. Specifically, if “Yes” is selected in step S3-39 shown in FIG. 28, the step may proceed to step S3-65 shown in FIG. 58.

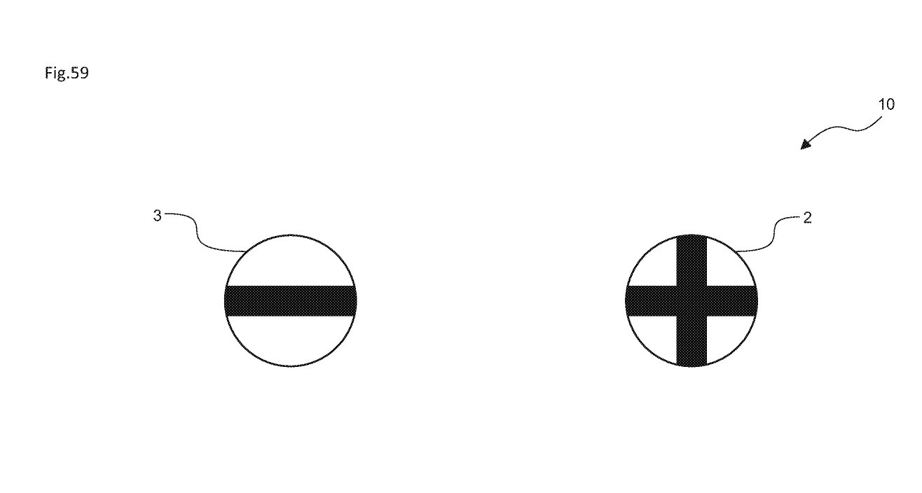

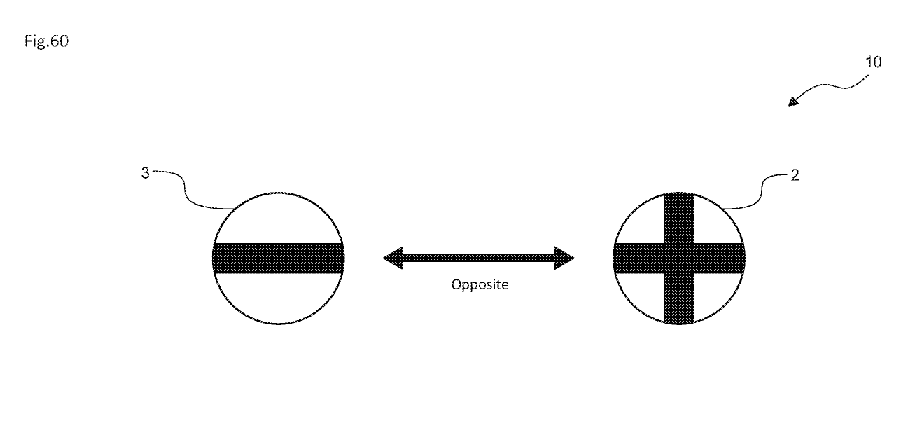

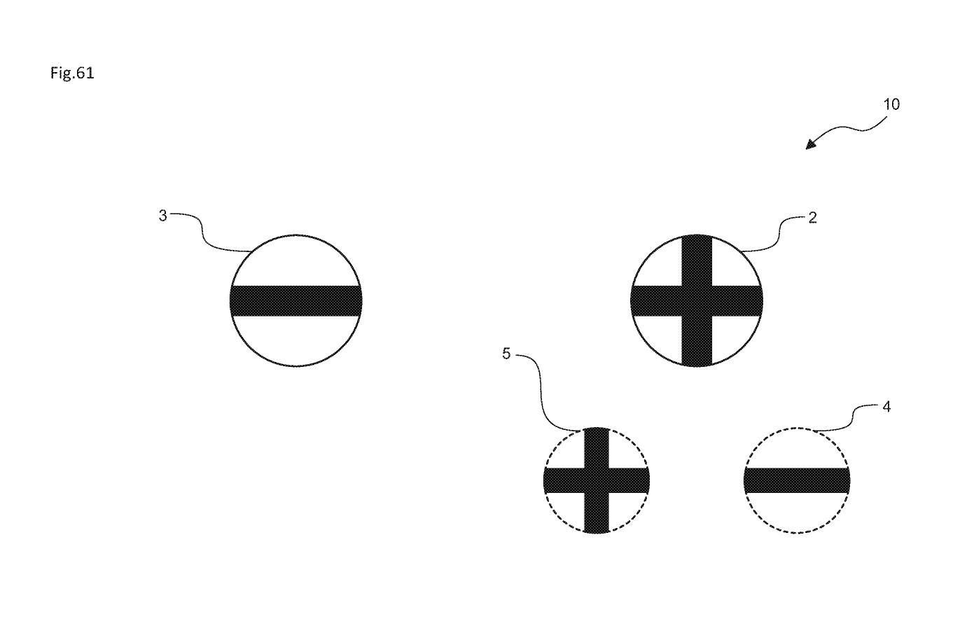

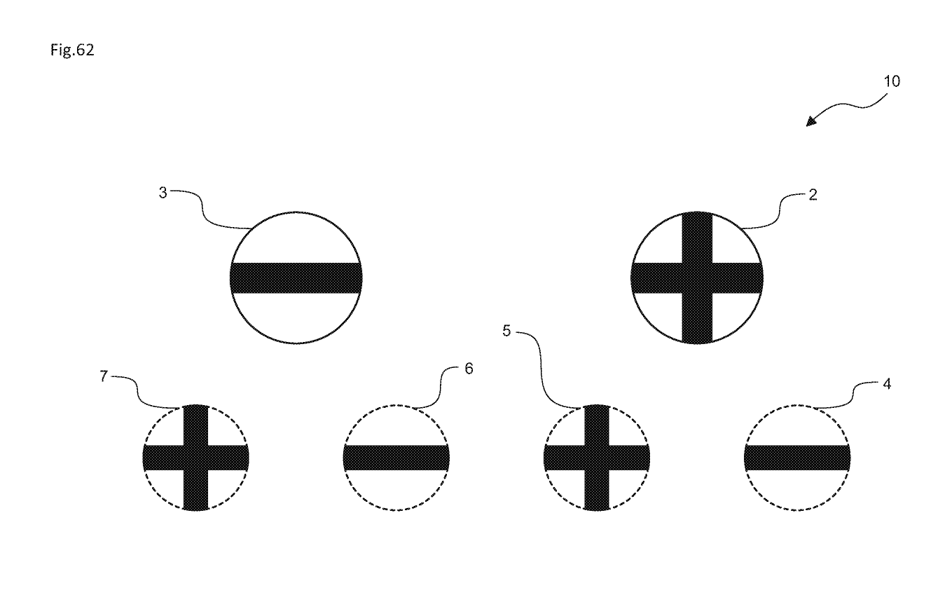

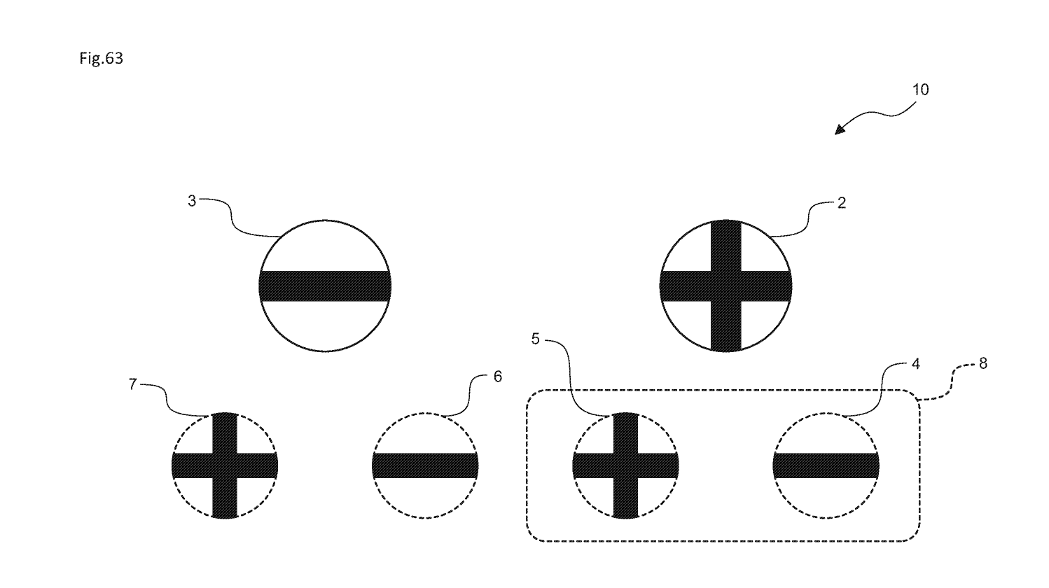

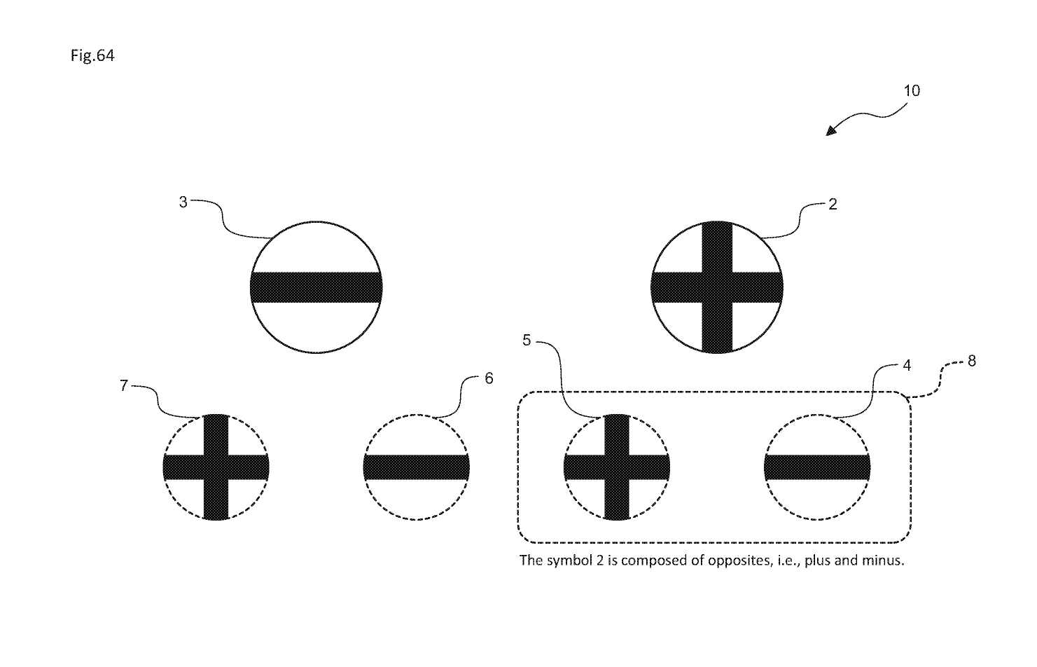

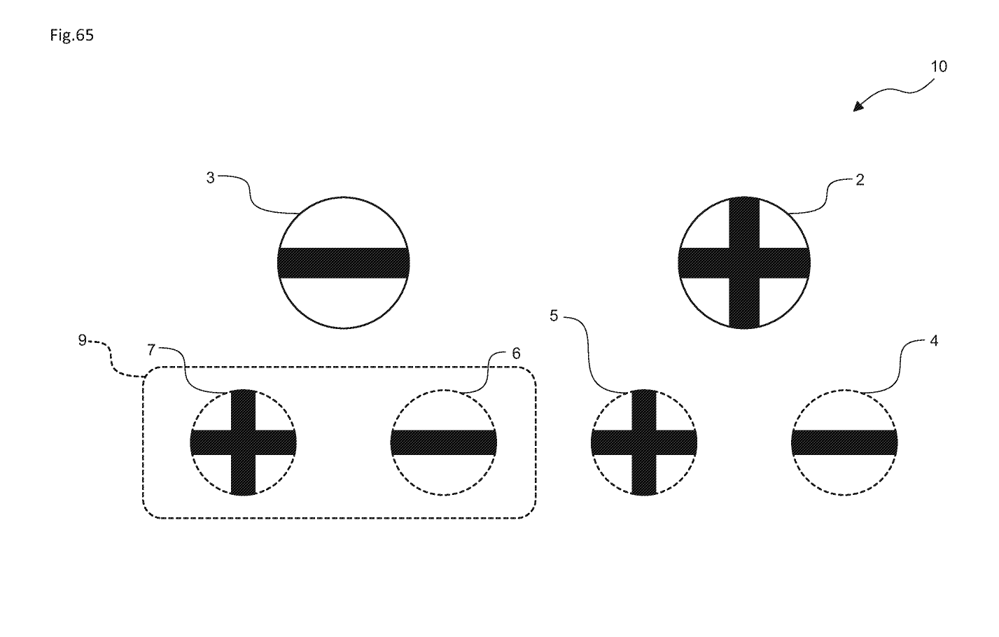

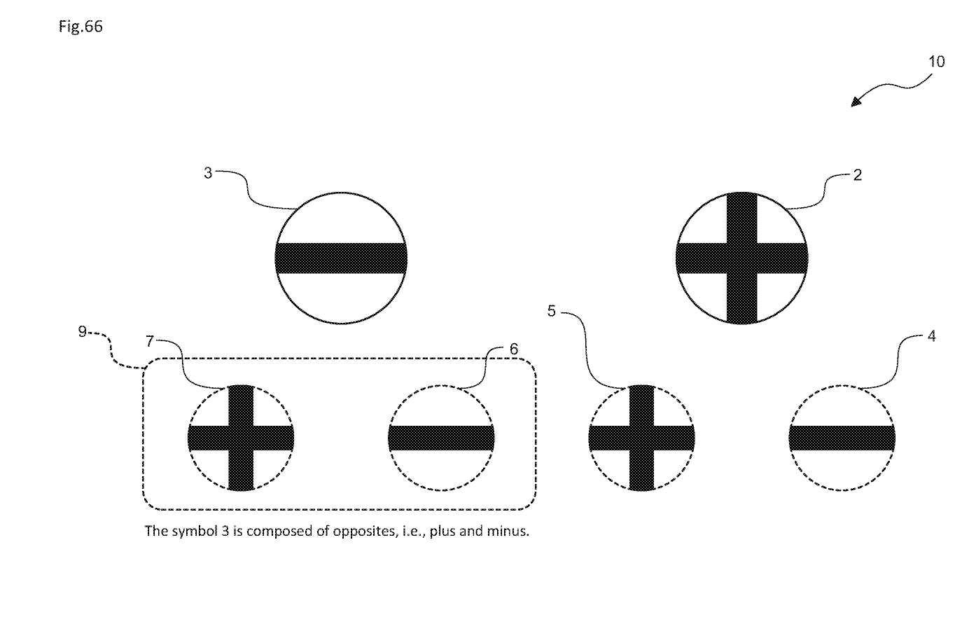

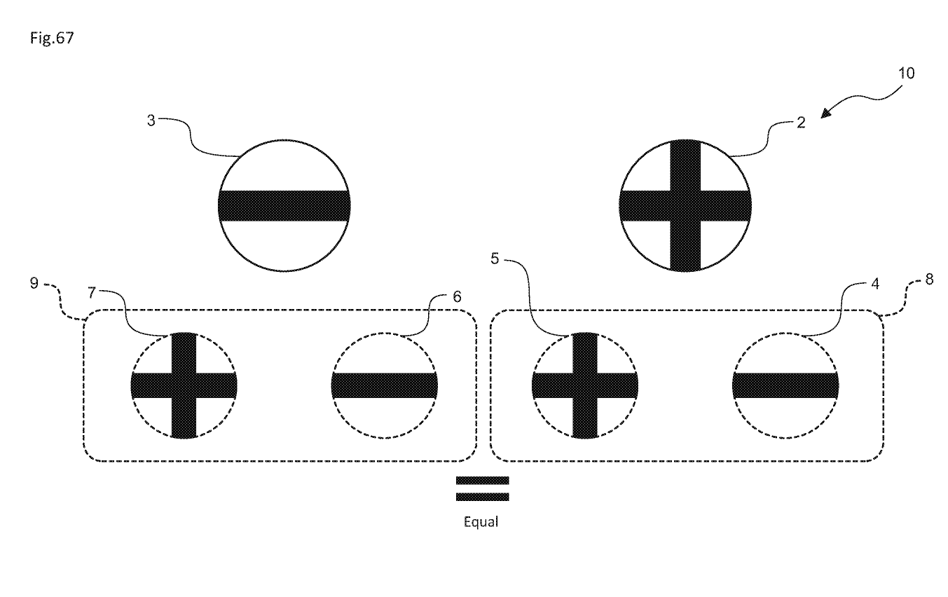

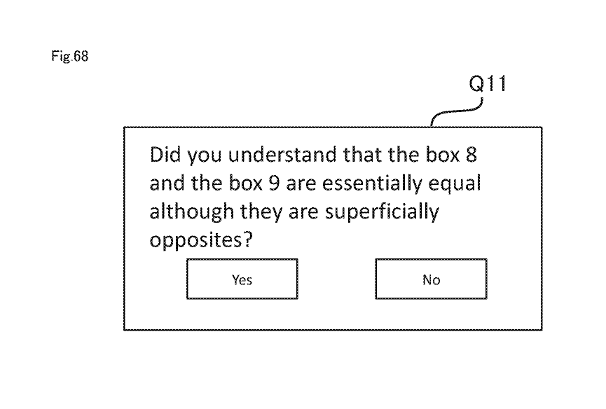

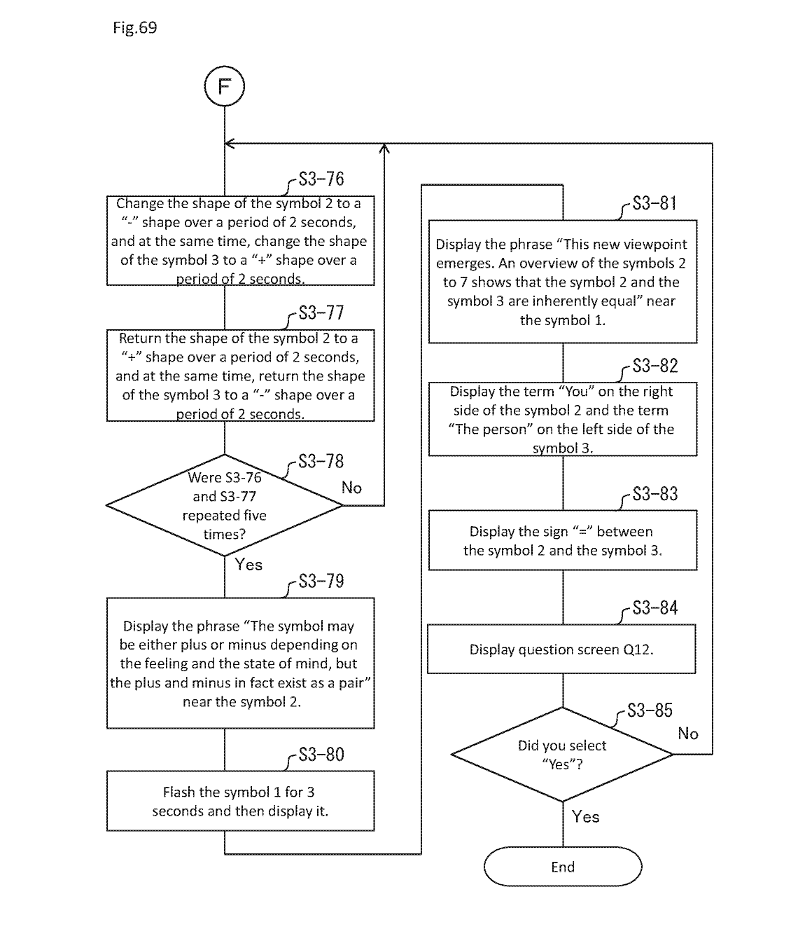

Subsequently, in step S3-65 shown in FIG. 58, in a state where the set 10 is displayed, the first symbol 1 and the fourth to seventh symbols 4 to 7 are flashed for 2 seconds and then not displayed, thereby leading to the state shown in FIG. 59. Further, as shown in FIG. 60, a double-headed arrow is flashed with the term “opposite” between the second symbol 2 and third symbol 3 for 3 seconds (step S3-66). Thereafter, as shown in FIG. 61, symbols having “+” and “−” shapes are displayed from behind the symbol 2 and moved to the positions of the fifth symbol 5 and the fourth symbol 4, respectively (step S3-67). Likewise, as shown in FIG. 62, symbols having “+” and “−” shapes are displayed from behind the symbol 3 and moved to the positions of the seventh symbol 7 and the sixth symbol 6, respectively (step S3-68). Subsequently, as shown in FIG. 63, the fourth symbol 4 and the fifth symbol 5 are enclosed with a dotted-line box 8 (step S3-69). Furthermore, as shown in FIG. 64, the phrase “The symbol 2 is composed of opposites, i.e., plus and minus” is displayed just below the box 8 for 3 seconds (step S3-70). Likewise, as shown in FIG. 65, the sixth symbol 6 and the seventh symbol 7 are enclosed with a dotted-line box 9 (step S3-71). Furthermore, as shown in FIG. 66, the phrase “The symbol 3 is composed of opposites, i.e., plus and minus” is displayed just below the box 9 for 3 seconds (step S3-72). Then, as shown in FIG. 67, the sign “=” is displayed between the box 8 and the box 9 for 3 seconds (step S3-73). Subsequently, question screen Q11 shown in FIG. 68 is displayed (step S3-74). The question screen Q11 includes the message “Did you understand that the box 8 and the box 9 are essentially equal although they are superficially opposites?” and “Yes” and “No” buttons. If the examinee selects “Yes” (“Yes” in step S3-75), the step proceeds to step S3-76 shown in FIG. 69. If the examinee selects “No” (“No” in step S3-75), steps S3-65 to S3-74 are repeated.

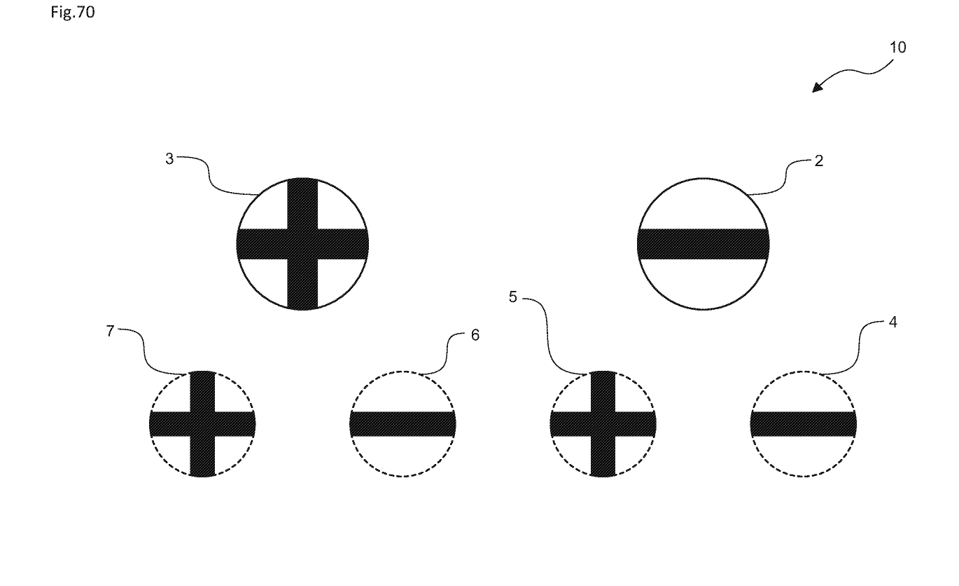

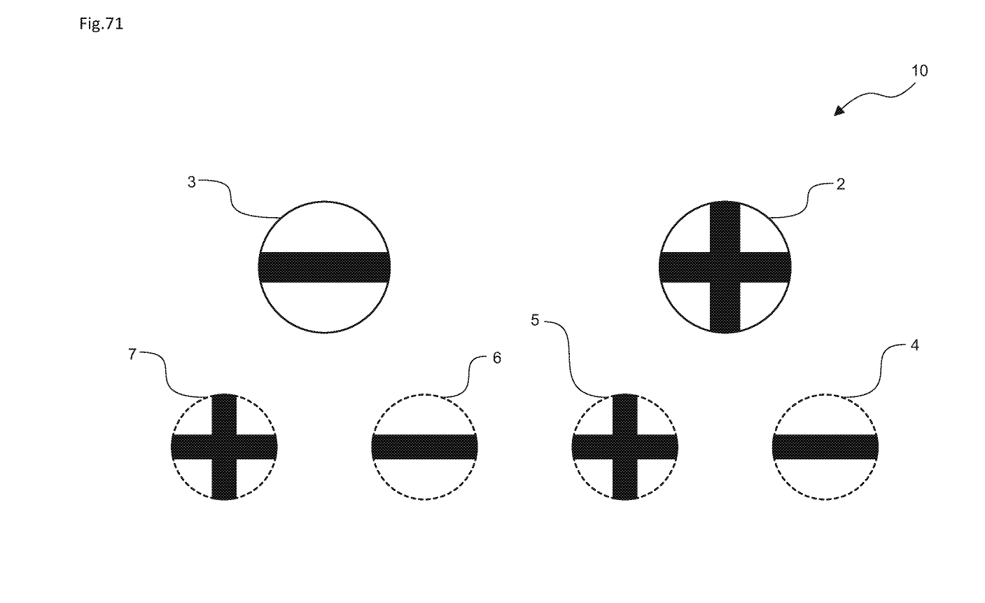

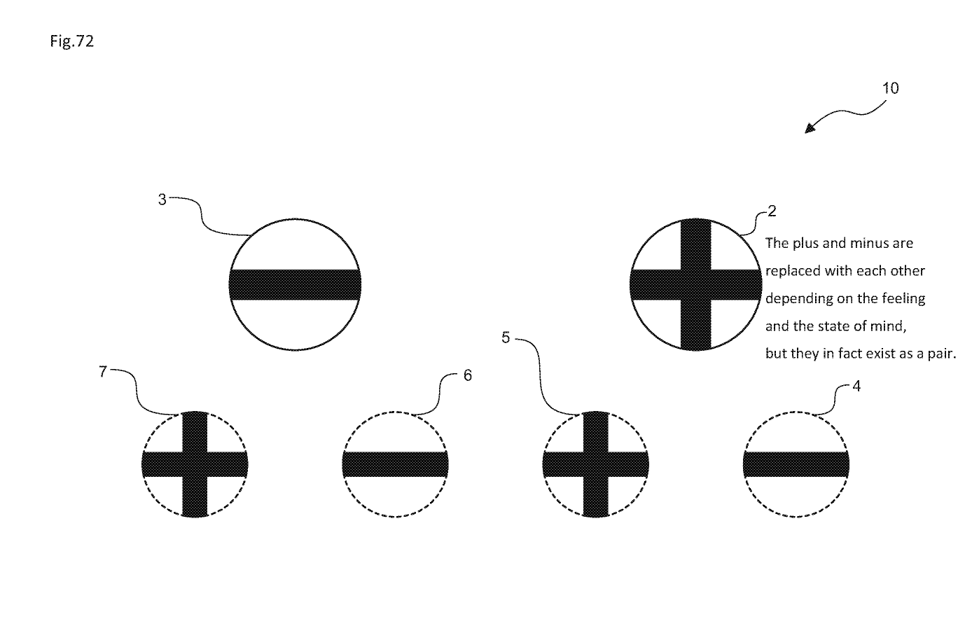

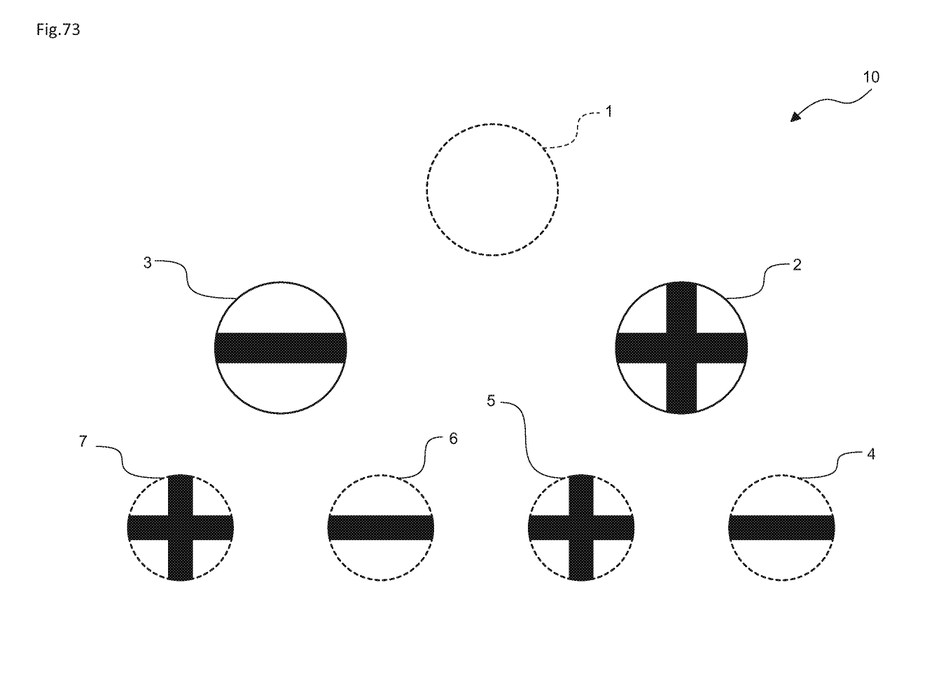

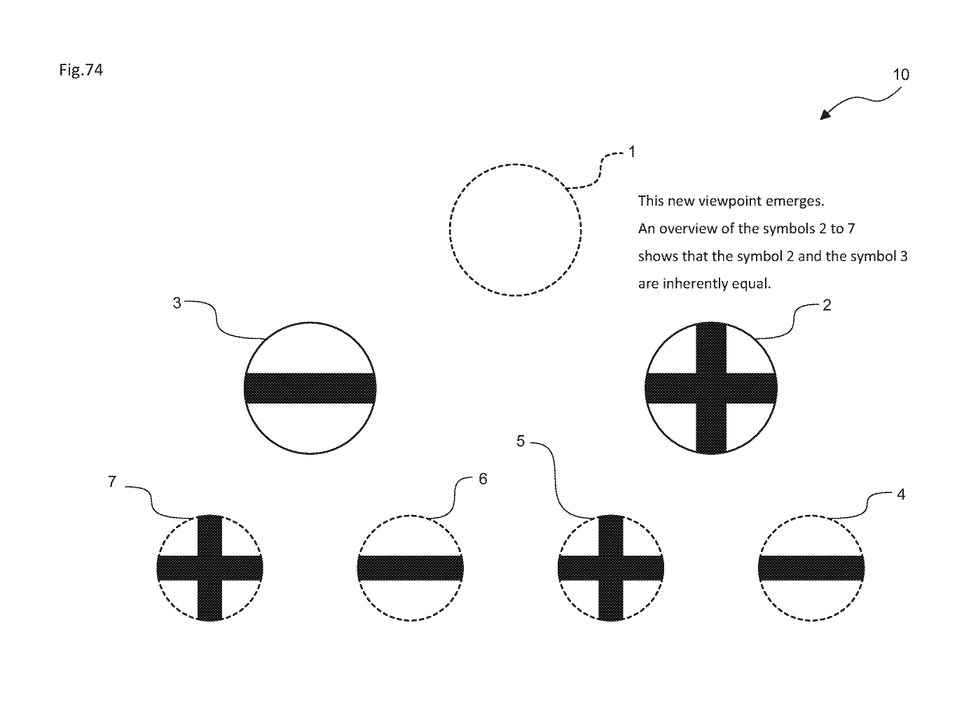

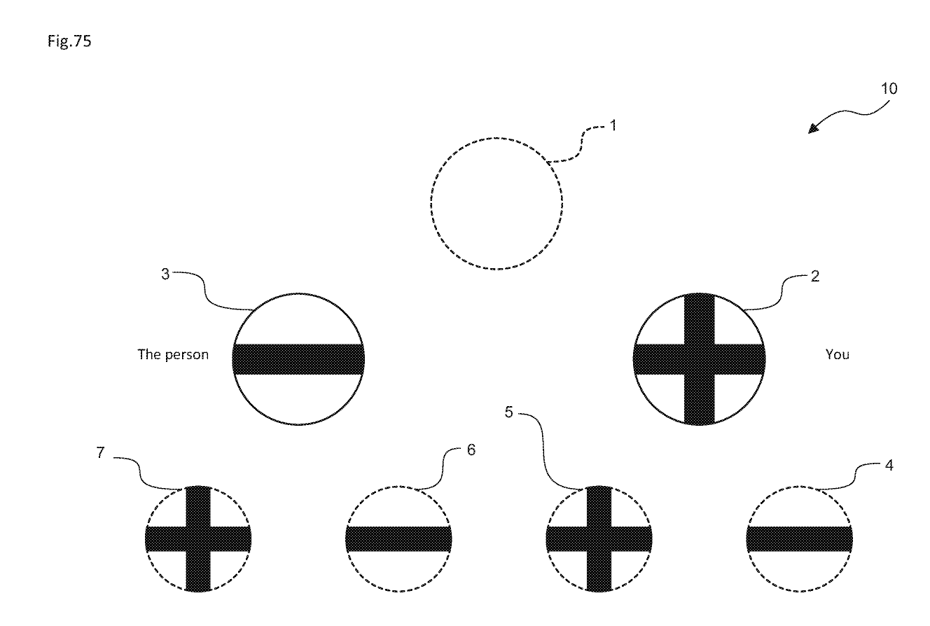

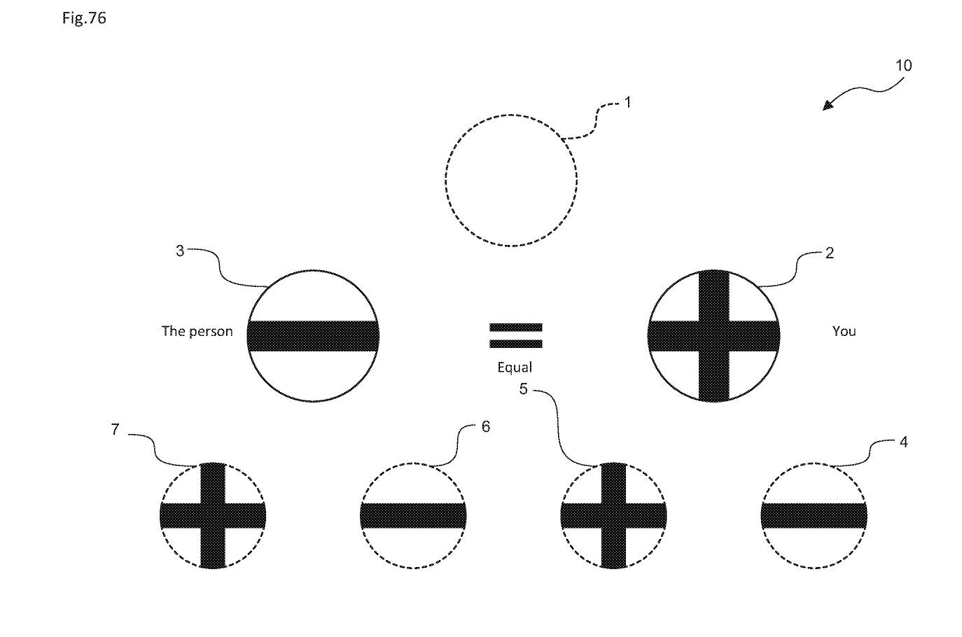

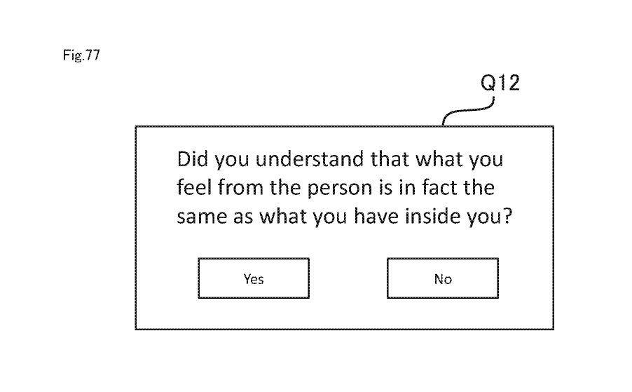

Subsequently, in step S3-76 shown in FIG. 69, in a state where the second to seventh symbols 2 to 7 are displayed, the shape of the second symbol 2 is changed to a “−” shape over a period of 2 seconds, and at the same time, the shape of the third symbol 3 is changed to a “+” shape over a period of 2 seconds, as shown in FIG. 70 (step S3-76). Furthermore, as shown in FIG. 71, the shape of the second symbol 2 is returned to a “+” shape over a period of 2 seconds, and at the same time, the shape of the third symbol 3 is returned to a “−” shape over a period of 2 seconds (step S3-77). After steps S3-76 and S3-77 are repeated five times (“Yes” in step S3-78), the phrase “The symbol may be either plus or minus depending on the feeling and the state of mind, but the plus and minus in fact exist as a pair” is displayed near the symbol 2 (step S3-79). Subsequently, as shown in FIG. 73, the first symbol 1 is flashed for 3 seconds and then displayed (step S3-80). Thereafter, as shown in FIG. 74, the phrase “This new viewpoint emerges. An overview of the symbols 2 to 7 shows that the symbol 2 and the symbol 3 are inherently equal” is displayed near the first symbol 1 (step S3-81). Subsequently, as shown in FIG. 75, the term “You” is displayed on the right side of the symbol 2, and the term “The person” is displayed on the left side of the symbol 3 (step S3-82). Further, as shown in FIG. 76, the sign “=” is displayed between the symbol 2 and the symbol 3 (step S3-83). Subsequently, question screen Q12 shown in FIG. 77 is displayed (step S3-84). The question screen Q12 includes the message “Did you understand that what you feel from the person is in fact the same as what you have inside you?” and “Yes” and “No” buttons. If the examinee selects “Yes”

(“Yes” in step S3-85), step S3 shown in FIG. 6 is completed. If the examinee selects “No” (“No” in step S3-85), steps S3-76 to S3-84 are repeated.

A specific example of the procedure in step S3 in this embodiment is described above. A questionnaire or the like may be given to the examinee in advance to find out information regarding a specific cause of conflicts in the examinee's mind and the background of the examinee's life, and the information may be stored in the auxiliary storage device 103 of the computer 100; the computer 100 may specifically explain to the examinee the meanings represented by the shapes of the symbols, using expressions such as “warmth (+)” and “coldness (−).” A pair of concepts, i.e., a pair of feelings and a pair of states of mind, may be, for example, “independence” and “dependence,” and “optimism” and “pessimism,” in addition to “warmth” and “coldness.”

The examinee's unconsciousness and a blind spot in the examinee's mind respectively associated with the fourth symbol 4 and the fifth symbol 5 are concepts with two polarities that produce conflicts in the examinee's mind. Everyone has these concepts in the mind. The unconsciousness and blind spot are in constant conflict in the mind, as in the fourth symbol 4 and the fifth symbol 5. In the example given above, when the examinee is feeling coldness in a boss at a workplace, there are concepts with two polarities inside the examinee's mind, i.e., “warmth (+)” and “coldness (−),” and the “warmth (+)” wins out over the conflict in the mind, allowing the examinee's thought (the second symbol 2) to identify with “warmth (+).” As a result, a phenomenon appears in which the examinee feels “coldness (−)” in the object of the examinee's thought. The “warmth (+)” is identified with by the examinee, which is why the examinee does not usually recognize their own “warmth” (blind spot =the fifth symbol 5). At the same time, the examinee's “coldness (−)” is below consciousness, and the examinee cannot recognize that the coldness is the examinee's own state. The coldness is put into a state of unconsciousness (the fourth symbol 4). The “coldness” (the third symbol 3) that the examinee feels in a boss is the “coldness (−)” in the examinee in a state of unconsciousness projected onto the object in front of the examinee. Specifically, the feeling from the object (cold boss) of the thought recognized by the examinee is the examinee's own unconsciousness exposed to the examinee's thought.

As described above, a phenomenon in which the thought (+) identified with by the examinee (subject) is reversed and projected onto others (object: (−)) in front of the examinee is defined as a phenomenon of “reversal by identification.” In the above example, the “warmth (+)” identified with by the examinee (subject) is reversed and projected as coldness (−) onto the boss before the examinee (object), who is an object of the examinee's thought.

As described above, the computer 100 explains to the examinee that the blind spot in the examinee's mind associated with the fifth symbol 5 represents a concept identified with by the examinee's thought associated with the second symbol 2, and at the same time, produces the examinee's unconsciousness associated with the fourth symbol 4, and that the object of the thought associated with the third symbol 3 is a projection of the examinee's unconsciousness associated with the fourth symbol 4. In the set 10, the third and fourth symbols 3 and 4 have a common shape, and the fifth symbol 5 has a shape representing a concept in contrast with that of the third and fourth symbols 3 and 4. Therefore, it is easy to understand the relationship between the symbols, based on their shapes, and thus to understand the recognition system represented by the set 10.

The computer 100 also explains to the examinee a case in which the examinee's thought (subject) and an object (object) of the examinee's thought are reversed. In this case, the computer 100 explains to the examinee that a blind spot in the object's mind associated with the sixth symbol 6 is identified with by the object's thought associated with the third symbol 3, and at the same time, produces the object's unconsciousness associated with the seventh symbol 7. Additionally, the computer 100 explains to the examinee that, the examinee's thought associated with the second symbol 2 is a projection of the object's unconsciousness associated with the seventh symbol 7, according to the phenomenon of “reversal by identification.” Specifically, the computer 100 explains that unconsciousness and a blind spot also exist inside the mind of the object of the examinee's thought, as with the inside of the examinee's mind as described above, although the polarities are different. When the set 10 does not include the sixth and seventh symbols 6 and 7, what is described in this paragraph may be omitted.

In this embodiment, it is preferable that the second and third steps (S2 and S3) are performed while confirming the degree of understanding of the examinee through interaction between the computer 100, which is a robot with artificial intelligence, and the examinee. Specifically, the computer 100 gives an explanation to the examinee via the audio output device 104 and receives the examinee's speech via the sound collection device 105, and performs speech recognition processing, thereby determining the examinee's degree of understanding. Alternatively, the examinee's degree of understanding may be determined by showing on the display device 106 a question as to whether the examinee has understood, and having the examinee answer the question through the input device 107. The second and third steps (S2 and S3) may be performed along predetermined interaction patterns, without performing speech recognition processing.

The time required for performing the third step (S3) is different for each individual. Some examinees may understand the recognition system with step S3 performed only once, while others may require step S3 to be performed multiple times.

In the third step (S3), when the examinee understands the recognition system, the conflict in the examinee's mind can be resolved to achieve balance of the examinee's mind. Specifically, the unconsciousness and blind spot in the examinee's mind are exposed in the examinee's thought.

Neither the unconsciousness nor the blind spot in the mind are recognized by the examinee; however, in the third step (S3), when the examinee understands that the unconsciousness of the examinee is projected onto the object of the examinee's thought before the examinee, based on the phenomenon of “reversal by identification,” the examinee can recognize the unconsciousness present in the examinee's own mind and allow it to be exposed.

In other words, the direction of the examinee's thought toward the object (the third symbol 3) is reversed so that the examinee's thought is directed to the examinee's own unconsciousness (the fourth symbol 4). For example, when the “coldness in a boss” is considered as the feeling from the object of the examinee's thought, the “coldness in a boss” is the coldness in the examinee's unconsciousness projected onto the boss. When the direction of the thought regarding the feeling of coldness is reversed toward the examinee him/herself, the examinee's unconsciousness (coldness) is allowed to be exposed in the examinee's thought. Along with this, the examinee also recognizes the “warmth,” i.e., a blind spot in the mind, which is identified with by the examinee him/herself. This is specifically a state in which the blind spot in the examinee's mind is exposed in thought.

When the examinee's unconsciousness (the fourth symbol 4) and the blind spot in the examinee's mind (the fifth symbol 5) are exposed in the examinee's thought, they fuse with each other as with the positive and negative charges of electrical energy, and are pair-annihilated. As a result, the thought associated with the second symbol 2 that caused a conflict in the examinee's mind also disappears. The first symbol 1 is located at the top of the set 10, which includes bisymmetrically positioned first to seventh symbols 1 to 7. The first symbol 1 has a shape and pattern that are not biased to either “+” or “−.” Specifically, the first symbol 1 indicates that the examinee's thought (the second symbol 2) biased toward “+” disappears, and the examinee's viewpoint is shifted from the second symbol 2 to a position (the first symbol 1) overviewing the entire recognition system represented by the set 10. Thus, when the examinee understands that the fourth symbol 4 (−) and the fifth symbol 5 (+) are pair-annihilated, and that the examinee's viewpoint is shifted from the second symbol 2 to the first symbol 1, the examinee's unconsciousness and the blind spot in the examinee's mind are easily pair-annihilated. Specifically, the first symbol 1 shows a balanced state of mind with no judgment of whether it is cold or not cold, or whether it is warm or not warm. More specifically, the first symbol 1 overviews the second and third symbols 2 and 3 simultaneously, and further overviews the second to seventh symbols 2 to 7, as well. That is, the first symbol 1 represents a state of an examinee who has gained a new viewpoint and become relaxed, being free from the two polarities of “approval (+)” and “denial (−),” i.e., (+) and (−), in the mind.

When the examinee's unconsciousness (the fourth symbol 4) disappears, a projection of the examinee's unconsciousness, i.e., the feeling from the object of the examinee's thought (the third symbol 3), also disappears at the same time. As a result, balance of the mind of the examinee is achieved.

For example, the “coldness in a boss” is assumed to be a feeling from the object of the thought. In this case, when the conflict in mind is resolved, the examinee no longer feels coldness in the boss, and no longer experiences stress from the boss.

The same applies to a case in which the subject and the object are reversed. Specifically, when the thought that causes a conflict in the examinee's mind and that is associated with the second symbol 2 disappears, the third symbol 3, which is an object of the examinee's thought also disappears at the same time. At this time, the unconsciousness in the mind associated with the seventh symbol 7 also disappears, and along with this, the blind spot associated with the sixth symbol 6 also disappears.

When the sixth and seventh symbols 6 and 7 are respectively associated with a blind spot and unconsciousness of the object of the examinee's thought, the examinee furthermore easily understands the recognition system represented by the set 10.

### 4. Others

An embodiment of the present invention is described above. However, the present invention is not limited to the above embodiment, and various modifications may be made without departing from the spirit of the present invention.

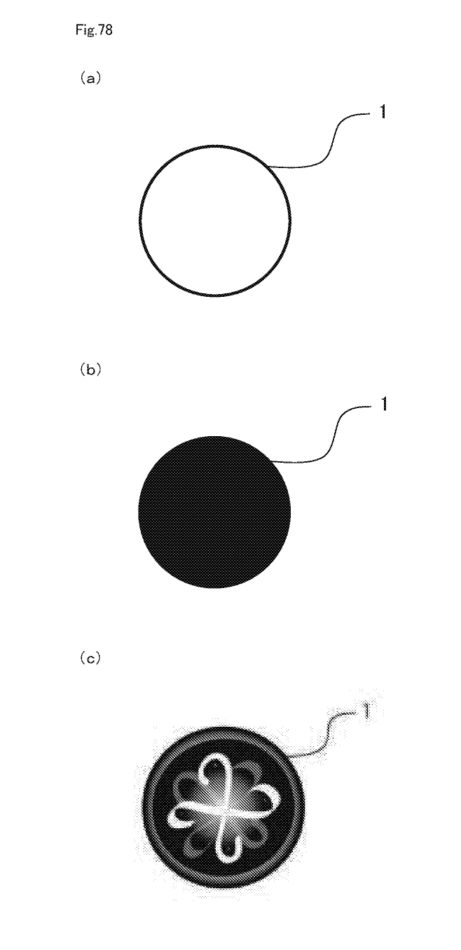

In the above embodiment, the first symbol 1 is a dotted-line circular frame; however, the first symbol 1 may be, for example, a white circle as shown in FIG. 78(a) or a black circle as shown in FIG. 78(b), or have a shape in which four pairs of Möbius strips are combined as shown in FIG. 78(c).

The above embodiment is an example in which the object of the examinee's thought is a boss of the examinee; however, the object of the examinee's thought is not limited thereto. The object of the thought is not particularly limited and may be a parent, spouse, friend, son, etc., of the examinee as long as the object is a cause of a conflict in the examinee's mind.

### 4-1. Modification 1

Furthermore, the object of the examinee's thought is not necessarily a person. For example, when an examinee feels “unreasonableness” against a community to which the examinee belongs, the “community” serves as the object of the thought of the examinee. The following describes an example in which the method of the present invention is applied to such an examinee, based on FIG. 6.

First, in step S1, the computer 100 makes an examinee visualize the set 10. Then, in step S2, the computer 100 applies the symbols of the set 10 to an examinee's feeling and a state of the examinee's mind. Steps S1 and S2 are the same as the steps used in the embodiment described above.

Subsequently, in step S3, the computer 100 makes the examinee understand the recognition system represented by the set 10, based on the contrasting concepts between the second symbol 2 and the third symbol 3, and between the fourth symbol 4 and the fifth symbol 5. Specifically, the computer 100 explains the following matters to the examinee.

The examinee's mind contains concepts with two polarities, such as “justice (+)” and “unreasonableness (−),” which produce a conflict. In the conflict, the “justice” wins out over the “unreasonableness,” and the examinee's thought (the second symbol 2) identifies with the “justice.” Thus, the examinee is not usually aware of the “justice” inside his/her mind, and the “justice” serves as a blind spot associated with the fifth symbol 5. At the same time, the “unreasonableness” in the mind of the examinee is below the examinee's consciousness; thus, the examinee does not recognize that the “unreasonableness” is the examinee's own state, and the “unreasonableness” is put into a state of unconsciousness associated with the fourth symbol 4. The examinee's “unreasonableness” feeling against the community (the third symbol 3) is the “unreasonableness” in the examinee in the state of unconsciousness and is projected onto the object of the examinee's thought. Specifically, the “justice” that is identified with by the examinee is projected onto the object of the examinee's thought as “unreasonableness,” based on the phenomenon of “reversal by identification.”

When the examinee understands the recognition system represented by the set 10 with the above explanation, the examinee's unconsciousness and a blind spot in the examinee's mind becomes exposed in the examinee's thought. Specifically, the examinee reverses the direction of the thought regarding the “unreasonableness” feeling against the community, so that it is directed toward the examinee him/herself. The examinee then becomes aware of his/her own unconsciousness (unreasonableness), and the unconsciousness becomes exposed in the thought. Along with this, the examinee recognizes the “justice,” which is a blind spot in mind identified with by the examinee him/herself, and the blind spot in the examinee's mind is also exposed in the examinee's thought.

In this manner, the examinee's unconsciousness (the fourth symbol 4) and the blind spot in the examinee's mind (the fifth symbol 5) are pair-annihilated, and as a result, the thought that causes a conflict in the examinee's mind associated with the second symbol 2 also disappears. At the same time, the feeling from the object of the examinee's thought, i.e., a projection of the examinee's unconsciousness (the third symbol 3), also disappears. It is thereby possible for the examinee to achieve balance of the mind. The examinee thus no longer feels unreasonableness against the community.

### 4-2. Modification 2

In another embodiment, the method according to the present invention is applied to an examinee who has a sense of inferiority to others as described below.

First, in step S1, the computer 100 makes an examinee visualize the set 10 by showing the examinee the set 10. Then, in step S2, the computer 100 applies the symbols of the set 10 to an examinee's feeling and a state of the examinee's mind. The steps S1 and S2 are the same as the steps used in the embodiment described above.

Subsequently, in step S3, the computer 100 makes the examinee understand the recognition system represented by the set 10, based on the contrasting concepts between the second symbol 2 and the third symbol 3, and between the fourth symbol 4 and the fifth symbol 5. Specifically, the computer 100 explains the following matters to the examinee.

The examinee's mind contains concepts with two polarities, such as “sense of superiority (+)” and “sense of inferiority (−),” which produce a conflict. In the conflict, the “sense of superiority” wins out over the “sense of inferiority,” and the examinee's thought (the second symbol 2) identifies with the “sense of superiority.” Thus, the examinee is not usually aware of the “sense of superiority” inside his/her mind, and the “sense of superiority” serves as a blind spot associated with the fifth symbol 5. At the same time, the examinee's “sense of inferiority” is below the consciousness; thus, the examinee does not recognize that the “sense of inferiority” is the examinee's own state, and the “sense of inferiority” is put into a state of unconsciousness associated with the fourth symbol 4. The examinee's “sense of inferiority” feeling from the object (the third symbol 3) of the examinee's thought is the “sense of inferiority” in the examinee's unconsciousness projected onto the object of the examinee's thought.

When the examinee understands the recognition system represented by the set 10 with the above explanation, the examinee's unconsciousness and a blind spot in the examinee's mind become exposed in the examinee's thought. Specifically, the examinee reverses the direction of the thought regarding the “sense of inferiority” feeling from the object of the examinee's thought, so that it is directed to the examinee him/herself. The examinee then becomes aware of his/her own unconsciousness, and the unconsciousness becomes exposed in the thought. Along with this, the examinee recognizes the “sense of superiority,” which is a blind spot in mind identified with by the examinee him/herself, and the blind spot in the examinee's mind is also exposed in the examinee's thought.

In this manner, the examinee's unconsciousness (the fourth symbol 4) and the blind spot in the examinee's mind (the fifth symbol 5) are pair-annihilated, and as a result, the thought that causes conflict in the examinee's mind associated with the second symbol 2 also disappears. At the same time, the feeling from the object of the examinee's thought, i.e., a projection of the examinee's unconsciousness (the third symbol 3), also disappears. It is thereby possible for the examinee to achieve balance of the mind. The examinee no longer has a sense of inferiority to others.

### EXAMPLES

The following are Examples of the present invention. However, the present invention is not limited to these Examples.

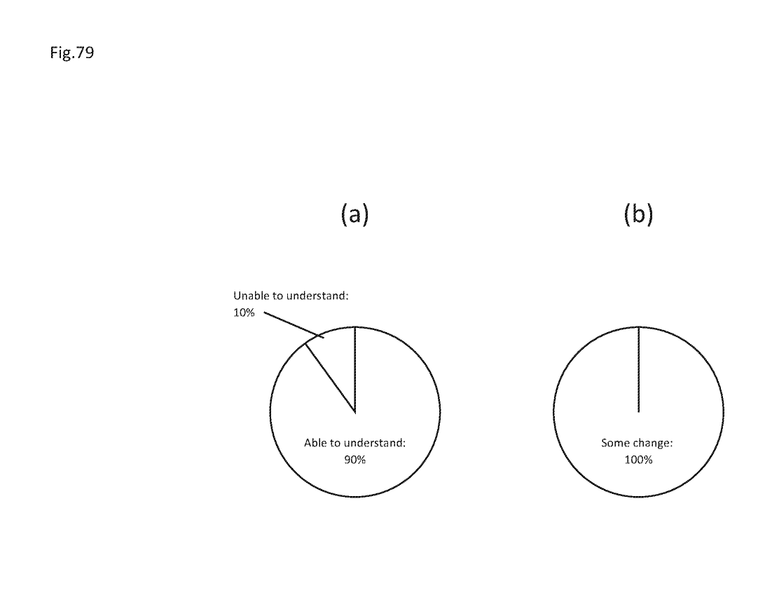

In the Example, the present invention was implemented for 265 examinees by using the computer 100. As the computer 100, a general-purpose personal computer in which the computer program according to the present invention was installed was used. After the present invention was implemented, a questionnaire survey was conducted on the examinees, and 10% (26 examinees) were randomly selected for counting. In the questionnaire, a question about changes in the examinees before and after the present invention was implemented was asked. FIGS. 79(a) and (b) show the answers to the question.
(a): Did you understand the recognition system represented by the set 10?
Yes: 90%, No: 10%
(b): If the answer was “Yes” to question (a), have you experienced any change?
Yes: 100%

As shown in FIG. 79(b), all of the examinees who answered that they had understood the recognition system represented by the set 10 felt that they had experienced changes between before and after the present invention was implemented. After understanding the recognition system represented by the set 10, the examinees at least recognized concepts having two polarities in their mind associated with the fourth and fifth symbols 4 and 5, and the concepts having two polarities were thus made exposed in their thought. Then, the unconsciousness and blind spot disappeared, allowing balance of the mind to be achieved.

### INDUSTRIAL APPLICABILITY

The present invention is suitably applied to fields in which problems in personal relationships (e.g., communication, love, marriage, and child raising) are solved, and can also be applied to fields of psychological medical treatment and epigenomes. The present invention is not limited to these and can be applied to solving any problems in humans, thus contributing to improvements in intellectual production activities.

### EXPLANATION OF REFERENCE NUMERALS

1 First symbol
2 Second symbol
3 Third symbol
4 Fourth symbol
5 Fifth symbol
6 Sixth symbol
7 Seventh symbol
8 Box
9 Box
10 Set of symbols
10a Set of symbols
10b Set of symbols
10c Set of symbols
100 Computer
101 CPU (processing device)
102 Memory (storage device)
103 Auxiliary storage device
104 Audio output device
105 Sound collection device
106 Display device
107 Input device
110 Computer program
111 Symbol data
112 Screen data
113 Dictionary data

1. A computer program configured to cause a computer including a processing device and a storage device to execute processing for achieving balance of the mind of an examinee, the computer program being configured to cause the computer to execute the following steps:
a first step of making the examinee visualize a set of symbols;
a second step of applying the symbols to a feeling of the examinee and a state of the examinee's mind; and
a third step of making the examinee understand a recognition system represented by the set.

2. The computer program according to claim 1, wherein the computer is a robot having artificial intelligence.

3. The computer program according to claim 1,
wherein the set includes:
a first symbol;
a second symbol located obliquely below the first symbol;
a third symbol that has a shape representing a concept in contrast with that of the second symbol, and that is a mirror image of the second symbol in terms of the position with respect to an imaginary vertical line passing through the first symbol;
a fourth symbol that has a shape in common with that of the third symbol and that is located obliquely below the second symbol; and
a fifth symbol that has a shape in common with that of the second symbol, and that is a mirror image of the fourth symbol in terms of the position with respect to an imaginary vertical line passing through the second symbol;
wherein in the second step, the second symbol is associated with the examinee's thought, the third symbol is associated with an object of the examinee's thought, the fourth symbol is associated with the examinee's unconsciousness, and the fifth symbol is associated with a blind spot in the examinee's mind; and
wherein in the third step, the examinee is made to understand the recognition system by an explanation based on contrasting concepts between the second symbol and the third symbol, and between the fourth symbol and the fifth symbol, the explanation being that the blind spot in the examinee's mind associated with the fifth symbol represents a concept identified with by the examinee's thought associated with the second symbol and produces the examinee's unconsciousness associated with the fourth symbol, and that the feeling from the object associated with the third symbol is a projection of the examinee's unconsciousness associated with the fourth symbol.

4. The computer program according to claim 3, wherein the second, fifth, and seventh symbols have a “+” shape while the third, fourth, and sixth symbols have a “−” shape.

5. The computer program according to claim 4, wherein the set further includes:
a sixth symbol that has a shape in common with that of the third symbol and that is located obliquely below the third symbol; and
a seventh symbol that has a shape in common with that of the second symbol, and that is a mirror image of the sixth symbol in terms of the position with respect to an imaginary vertical line passing through the third symbol;
wherein in the second step, the sixth symbol is associated with a blind spot in the object's mind, and the seventh symbol is associated with the object's unconsciousness; and
wherein in the third step, the examinee is given an explanation that the blind spot in the object's mind associated with the sixth symbol represents a concept identified with by the object's thought associated with the third symbol and produces the object's unconsciousness associated with the seventh symbol, and that the feeling from the examinee associated with the second symbol is a projection of the object's unconsciousness associated with the seventh symbol.
